(19) United States
(12) Patent Application Publication
(10) Pub. No.: US 2026/0148811 A1
(43) Pub. Date: May 28, 2026

(54) ELECTRONIC INFRASTRUCTURE FOR VIRTUALLY-ENABLED ON-LINE CONTENT GENERATION AND/OR STORAGE

(71) Applicant: ARH Technologies, LLC, Clearwater, FL (US)

(72) Inventor: Alan R. Hollander, Clearwater, FL (US)

(21) Appl. No.: 19/372,174

(22) Filed: Oct. 28, 2025

Related U.S. Application Data
(60) Provisional application No. 63/712,701, filed on Oct. 28, 2024.

(51) Int. Cl. G16H 10/20; G06F 9/451; G06Q 10/1093

(52) U.S. Cl. CPC G16H 10/20; G06F 9/451; G06Q 10/1093

(57) ABSTRACT

Example methods, apparatuses, and/or articles of manufacture are disclosed herein that may be utilized, in whole or in part, to facilitate and/or support one or more operations and/or techniques for electronic infrastructure for virtually-enabled on-line content generation and/or storage, such as implemented, in whole or in part, via one or more computing and/or communication networks and/or protocols.

### CROSS-REFERENCE TO RELATED APPLICATIONS

This application claims the benefit of and priority to U.S. Provisional Patent Application Ser. No. 63/712,701, titled “ELECTRONIC INFRASTRUCTURE FOR VIRTUALLY-ENABLED ON-LINE DATA ENTRY,” filed on Oct. 28, 2024, which is expressly incorporated herein by reference in its entirety.

### BACKGROUND

### 1. Field

The present disclosure relates generally to electronic infrastructures and/or computing environments and, more particularly, to electronic infrastructure for virtually-enabled on-line content generation and/or storage that may be used, at least in part, to facilitate and/or support more secure on-line content origination, confirmation, verification, etc., such as in privacy-regulated professional and/or other settings, for example, that may be implemented, in whole or in part, via one or more computing and/or communication networks and/or protocols.

### 2. Information

Distributed or like computing systems, including for particular privacy-regulated industries, such as, for example, the existing Health Insurance Portability and Accountability Act (HIPAA)-regulated patient digital intake platforms, patient management systems, or the like are often unable to adequately support the evolving needs of privacy-regulated professional or other practices. Many conventional systems rely on rigid, form-based input interfaces for providers to input data into static fields, leading to incomplete submissions, inconsistent data quality, increased follow-up tasks, etc. Architecturally, these or like systems are frequently fragmented, with separate modules or third-party applications handling different tasks, such as, for example, authentication, scheduling, messaging, form completion, data storage, etc. In some instances, such fragmentation may increase system latency, introduce synchronization errors, complicate backend operations by generating redundant Application Programming Interface (API) calls and duplicated records, or other issues. Moreover, many existing platforms lack support for multimodal input, such as natural language processing (NLP), voice commands, real-time image capture, etc., which may reduce accessibility and user engagement, particularly on client devices (e.g., mobile devices, kiosk interfaces, etc.). Backend infrastructures for these or like platforms also tend to be inflexible, using rigid data models and limited service orchestration, for example, which prevents these or like platforms from adapting dynamically to conversational workflows or integrating efficiently with external data sources, among other aspects. These aspect may result in higher administrative overhead, slower workflows, less effective use of computational resources, etc., ultimately preventing existing platforms from meaningfully improving the efficiency of privacy-regulated professional operations.

In parallel with these computing and/or communication challenges, privacy-regulated professional industries, such as dental, medical, legal, business or like practices continue to face significant time and resource inefficiencies in how they interact with clients and/or patients. For example, an oral surgeon may spend a substantial portion of each workday engaged in preoperative consultations, patient education, or follow-up discussions rather than performing surgical procedures. These interactions, while necessary, can reduce the time available for revenue-generating clinical activities and increase the workload of administrative and support staff. Staff members are often tasked with collecting patient data, verifying insurance information, explaining procedural details, and managing documentation—tasks that consume considerable time and attention. Even if digital tools are employed, the inefficiencies of current on-line platforms force much of this work to remain laborious, limiting potential gains in productivity and professional services to patients. As expectations shift toward faster, more seamless, and/or more automated digital experiences, the inability of existing systems to streamline these or like processes exacerbates operational inefficiencies and places additional strain on both practitioners and their staff. Greater flexibility and/or variety of on-line content generation and/or storage, such as to facilitate and/or support privacy-regulated professional or like industries, for example, may, therefore, be desirable.

### BRIEF DESCRIPTION OF THE DRAWINGS

Claimed subject matter is particularly pointed out and distinctly claimed in the concluding portion of the specification. However, both as to organization and/or method of operation, together with objects, features, and/or advantages thereof, it may best be understood by reference to the following detailed description if read with the accompanying drawings in which:

FIG. 1 is a schematic block diagram depicting an embodiment of an example system including one or more server computing devices and/or one or more Internet of Things (IoT)-type devices, in accordance with one or more embodiments;

FIG. 2 is a schematic block diagram depicting an embodiment of an example IoT-type device, in accordance with one or more embodiments;

FIG. 3 is an example flow diagram illustrating an example method for virtually-enabled online content generation, in accordance with one or more embodiments;

FIGS. 4A-4C are flow diagrams depicting example processes for virtually-enabled online content generation, in accordance with one or more embodiments;

FIG. 5 is another flow diagram depicting an example process for virtually-enabled online content generation, in accordance with one or more embodiments;

FIGS. 6A-6B is yet another flow diagram depicting an example process for virtually-enabled online content generation, in accordance with one or more embodiments;

FIG. 7 is an illustration depicting example diagrams depicting example aspects for virtually-enabled online content generation, in accordance with one or more embodiments;

FIG. 8 is a schematic block diagram illustrating an example computing environment, in accordance with one or more embodiments;

FIGS. 9A-9C are illustrations depicting an example graphical user interface (GUI) for virtually-enabled online content generation, in accordance with one or more embodiments.

Reference is made in the following detailed description to accompanying drawings, which form a part hereof, wherein like numerals may designate like parts throughout that are corresponding and/or analogous. It will be appreciated that the figures have not necessarily been drawn to scale, such as for simplicity and/or clarity of illustration. For example, dimensions of some aspects may be exaggerated relative to others, one or more aspects, properties, etc. may be omitted, such as for ease of discussion, or the like. Further, it is to be understood that other embodiments may be utilized, in whole or in part. Furthermore, structural and/or other changes may be made without departing from claimed subject matter. References throughout this specification to “claimed subject matter” refer to subject matter intended to be covered by one or more claims, or any portion thereof, and are not necessarily intended to refer to a complete claim set, to a particular combination of claim sets (e.g., method claims, apparatus claims, etc.), or to a particular claim. It should also be noted that directions and/or references, for example, such as up, down, top, bottom, and so on, may be used to facilitate discussion of drawings and are not intended to restrict application of claimed subject matter. Therefore, the following detailed description is not to be taken to limit claimed subject matter and/or equivalents.

### DETAILED DESCRIPTION

References throughout this specification to one implementation, an implementation, one embodiment, an embodiment, and/or the like means that a particular feature, structure, characteristic, and/or the like described in relation to a particular implementation and/or embodiment is included in at least one implementation and/or embodiment of claimed subject matter. Thus, appearances of such phrases, for example, in various places throughout this specification are not necessarily intended to refer to the same implementation and/or embodiment or to any one particular implementation and/or embodiment. Furthermore, it is to be understood that particular features, structures, characteristics, and/or the like described are capable of being combined in various ways in one or more implementations and/or embodiments and, therefore, are within intended claim scope. In general, of course, as has always been the case for the specification of a patent application, these and other issues have a potential to vary in a particular context of usage. In other words, throughout the disclosure, particular context of description and/or usage provides helpful guidance regarding reasonable inferences to be drawn; however, likewise, “in this context” in general without further qualification refers at least to the context of the present patent application.

Distributed or like computing systems, including for particular privacy-regulated industries, such as, for example, the existing HIPAA-regulated patient digital intake platforms, patient management systems, or the like are often unable to adequately support the evolving needs of privacy-regulated professional or other practices. Many conventional systems rely on rigid, form-based interfaces for providers to input data into static fields, leading to incomplete submissions, inconsistent data quality, increased follow-up tasks, etc. Architecturally, these or like systems are frequently fragmented, with separate modules or third-party applications handling authentication, scheduling, messaging, form completion, data storage, etc. In some instances, such fragmentation increases system latency, introduces synchronization errors, complicates backend operations by generating redundant API calls and duplicated records, or other issues. Moreover, many existing platforms lack support for multimodal input such as NLP, voice commands, real-time image capture, etc., which may reduce accessibility and user engagement, particularly on client devices (e.g., mobile devices, kiosk interfaces, etc.). Backend infrastructures for these or like platforms also tend to be inflexible, using rigid data models and limited service orchestration, for example, which prevents these or like platforms from adapting dynamically to conversational workflows or integrating efficiently with external data sources, among other aspects. These aspects may result in higher administrative overhead, slower workflows, less effective use of computational resources, etc., ultimately preventing existing platforms from meaningfully improving the efficiency of privacy-regulated professional operations.

In parallel with these computing challenges and/or communication challenges, privacy-regulated professional offices such as dental, medical, legal, business or like practices continue to face significant time and resource inefficiencies in how they interact with clients and/or patients. For example, an oral surgeon may spend a substantial portion of each workday engaged in preoperative consultations, patient education, or follow-up discussions rather than performing surgical procedures. These interactions, while necessary, can reduce the time available for revenue-generating clinical activities and increase the workload of administrative and support staff. Staff members are often tasked with collecting patient data, verifying insurance information, explaining procedural details, and managing documentation—tasks that consume considerable time and attention. Even if digital tools are employed, the inefficiencies of current platforms force much of this work to remain laborious, limiting the potential gains in productivity and professional services to patients. As patient expectations shift toward faster, more seamless, and/or more automated digital experiences, the inability of existing systems to streamline these processes exacerbates operational inefficiencies and places additional strain on both practitioners and their staff. Greater flexibility and/or variety of on-line content generation and/or storage, such as to facilitate and/or support privacy-regulated professional or like industries, for example, may, therefore, be desirable.

As mentioned, these or like challenges may be attributable to other types of professions, and an oral surgeon office is merely an example of a particular profession that may encounter these or like challenges, and is used herein for purposes of explanation and/or for ease of discussion.

As discussed more fully below, one or more embodiments may include, for example, a Virtual Consultation Infrastructure (VCI) that may be based, at least in part, on one or more computing processes tailored to address, at least in part, one or more challenges, which may include those mentioned above, among others. As utilized herein, the VCI may include example methods, apparatuses, and/or articles of manufacture disclosed herein that may be implemented, in whole or in part, using one or more computing devices to facilitate and/or support one or more operations, approaches, and/or techniques for virtually-enabled on-line data entry that may be used, at least in part, to facilitate and/or support one or more virtual intake processes that may include on-line form generation, virtual consultation, informed consent origination, confirmation, and/or verification, etc. for professional or other settings, such as dental, medical, business, or the like. As explained in more detail below, VCI may comprise machine-learning and/or AI aspects, in one or more embodiments.

For example, in an embodiment, a host application running on a client device, such as a mobile device, laptop computer, desktop computer, tablet device, etc. as part of a VCI may implement one or more special-purpose computer-readable instructions to generate a graphical representation (e.g., a two-dimensional icon, three-dimensional on-screen image, etc.) on a suitable GUI to function as a digital intake assistant, customizable or otherwise, designed to reflect a particular identity, character, likeness, etc. in a specific virtual environment. In some implementations, such a graphical representation may be used interchangeably with the term “avatar,” and may be virtually-enabled, such as implemented in connection with one or more tailored datasets trained and deployed for one or more operations and/or processes described herein. At times, such a virtually-enabled graphical representation may be part of a VCI's AI-assisted avatar, just to illustrate one possible implementation.

In some implementations, a VCI can display, on a user device, an AI-assisted avatar. The computer process for such an avatar can generate a prompt (e.g., audibly, on-screen, haptically, etc.) for initiation of an electronic process for content generation. The online content can be partitioned on a screen of a client device according to one or more predefined portions. The VCI can process an input in response to the computer prompt assign a value corresponding to a predefined field from within the input. The VCI can store the identified value in a particular database for use in one or more subsequent computing processes in connection with the VCU-enabled avatar.

As a way of illustration, a client device co-located with a particular user can display an AI-assisted avatar that audibly prompts a user to provide certain information. The client device can capture audio of a spoken user response, and the VCI can convert the audio to text using a speech-to-text model. The VCI can identify a value corresponding to the predefined field within the text and store the identified value in the field of the patient intake data table.

One or more embodiments described herein may be directed, in whole or in part, to an oral surgery practice, for example. However, other embodiments may be directed to other dental and/or medical practice-types and/or may be directed to any of a wide range of other professional practice-types (e.g., accounting, legal, finance, etc.), for example, and subject matter is not limited in scope in these respects.

In one or more embodiments, an AI-assisted infrastructure may support virtually-enabled on-line data entry that may be used, at least in part, to facilitate and/or support one or more virtual intake processes that may include on-line form generation, virtual consultation, informed consent origination, confirmation, and/or verification, etc., such as between an oral surgery practice and a patient, for example.

As way of illustration, a dentist may have a patient who is in need to the services of oral surgeon. For example, a dentist's office may send a referral to an oral surgery practice. Alternatively, of course, a patient may contact an oral surgery practice directly. In one or more embodiments, an electronic message indicating a referral and/or contact from patient may be obtained by a VCI that may be operated by and/or on behalf of an oral surgery practice, for example. In one or more embodiments, VCI may include one or more aspects that may be based, at least in part, on one or more trained machine learning/AI models, for example.

In one or more embodiments, VCI, at least in part in response to a referral and/or contact from a patient, may transmit an electronic message (e.g., text, email, phone call, etc.) to patient. In one or more embodiments, such a message may invite a patient to schedule a call (e.g., video session, etc.) via a provided link. In one or more embodiments, a link may be embedded in a text message, for example. In one or more embodiments, by following a provided link, a patient may be invited to schedule a telehealth session, such as may be established between a patient's cell phone or other electronic device and VCI, for example. In one or more embodiments, reminders may be sent to patient via electronic message, for example. Also, for example, VCI may provide notification to oral surgery practice staff and/or referring doctor's office, for example, of status regarding scheduling and/or at other steps along the way, in one or more embodiments.

In one or more embodiments, at a scheduled time, for example, a telehealth session may be established as described above. VCI may include an AI-generated avatar that may be referred to for this example as “Aria.” In one or more embodiments, Aria may be displayed to patient via patient's cell phone, for example, although other electronic devices may also be utilized, for example. In other embodiments, in some circumstances, an audio-only session may be established. Also, for example, Aria may generate speech and/or text, in one or more embodiments. See additional discussion below regarding accessibility, speech-to-text, and/or text-to-speech features for one or more embodiments.

In one or more embodiments, Aria may ask patient if they are ready to proceed. In one or more embodiments, a patient may indicate readiness to proceed by speaking an appropriate word, such as “start” and/or the like. In one or more embodiments, a patient may respond orally, with no need to type or write anything, for example. Of course, other embodiments may provide for typed input, although subject matter is not limited in scope in this respect.

In one or more embodiments, Aria may interact with a patient to gather information from patient and/or to provide information to the patient, for example. In one or more embodiments, a patient may provide responses orally, as previously alluded to, for example. Also, in one or more embodiments, Aria may interact with a patient to guide patient through various forms and/or to gather patient's approval, verification, informed consent, etc., for example. Along the way, in one or more embodiments, Aria may ask a patient to verify gathered information. For example, in one or more embodiments, Aria may display information to a patient as it is gathered to allow patient to verify. In one or more embodiments, patient-provided content (e.g., information, etc.) may be transcribed in real-time or near real-time, visible (and/or audible) to patient, for example. In one or more embodiments, Aria may allow a patient to make corrections (e.g., orally, etc.) if such corrections are indicated by patient, for example.

In one or more embodiments, a patient may submit, such as via interaction with Aria, signatures, insurance information, imaging (e.g., radiographs, etc.), identification documents, etc., for example. In one or more embodiments, Aria may allow patient to show insurance card, identification documents, etc. to Aria via patient's electronic device (e.g., cell phone, etc.), and Aria may gather an image of presented document(s), for example.

In one or more embodiments, Aria may read, show, and/or explain HIPAA forms to a patient, for example. In one or more embodiments, a number of different types of forms may be read, shown, explained, etc. In one or more embodiments, a patient may ask questions and/or Aria may answer questions, for example. In one or more embodiments, Aria's answers may be generated at least in part via one or more AI processes and/or approaches, for example. Also, in one or more embodiments, signatures, verification, approvals, and/or informed consent, for example, may be provided orally by patient via Aria, with patient checking and/or acknowledging signatures, verifications, approvals, and/or informed consent, for example. In one or more embodiments, time-stamped records may be kept, as discussed below, for example.

In one or more embodiments, one or more videos may be shown to patient, for example. In one or more embodiments, Aria may ask a patient if they are ready to proceed, and the patient may respond orally, for example. In one or more embodiments, videos may be shown to patient regarding any of a range of subjects. For example, if a patient is interacting with an oral surgery practice, Aria may show videos pertaining to dental implants, bone grafting, etc., as appropriate. In one or more embodiments, selection of videos to show to a patient may be made by Aria based, at least in part, on one or more AI-based processes and/or approaches, for example. Additionally, in one or more embodiments, a patient may select videos to watch from a menu displayed via Aria, for example. Of course, subject matter is not limited in scope in this respect.

Also, as mentioned, a patient may ask questions and/or Aria may provide answers and/or explanations (e.g., based at least in part on AI-based response generation engine, etc.), in one or more embodiments. In one or more embodiments, Aria may answer questions based at least in part on one or more trained machine-learning/AI models, for example. In one or more embodiments, by providing such videos, answers, and/or explanations, case acceptance may be boosted. In one or more embodiments, if Aria is unable to provide a satisfactory answer to a patient question, question may be flagged and/or VCI may provide an indication to office staff and/or the like so that the question may be addressed at a later time, such as at an in-person session, for example.

In one or more embodiments, Aria, and VCI more generally, for example, may track informed consent compliance and/or may reduce an amount of time a professional, such as an oral surgeon for the present example, may spend explaining subjects in-person, for example. In one or more embodiments, records may be kept via VCI. Such records may include digital video content showing patient providing approval, informed consent, etc., in one or more embodiments. In one or more embodiments, records may be time-stamped, for example.

In one or more embodiments, reminders may be provided to patient regarding future scheduled appointments, for example.

In one or more embodiments, a patient may be transitioned from Aria-driven virtual intake processes, for example, to in-person consultations with staff and/or with a doctor, for example. In one or more embodiments, via such in-person consultations, treatment plans (e.g., designed by a doctor, etc.) may be discussed, payments/finances may be arranged, and/or previously-unanswered questions may be addressed, for example. In one or more embodiments, as a result of previously-conducted, Aria-driven virtual intake processes, such in-person consultations may be reduced in number and/or duration. For example, a doctor may only spend a few minutes with a patient rather than more extended durations that may be the norm in other circumstances.

In one or more embodiments, VCI may facilitate scheduling of follow-on procedures. In one or more embodiments, for such follow-on procedures, similar virtual intake processes to go over forms, obtain informed consent, provide explanations and/or answer questions, etc., may occur in a manner similar to those explained above. Also, in one or more embodiments, Aria may follow-up with patient following a procedure to check on patient well-being and/or to answer questions, for example.

Potential advantages of a VCI-type approach, based at least in part on one or more trained machine-learning/AI models, for example, may include helping to ensure patients are engaged, informed, supported, and/or on track without unduly taking up staff's valuable time and/or valuable time of an oral surgeon, for example, and/or without having to hire additional staff. These are merely example possible advantages that may be provided via Aria and/or VCI. In one estimate, Aria and/or VCI may save eight hours of staff time and one hour of doctor time every day by use of such Aria-driven virtually-enabled on-line data entry that may be used, at least in part, to facilitate and/or support one or more virtual intake processes that may include on-line form generation, virtual consultation, informed consent origination, confirmation, and/or verification, etc., in one or more embodiments. With such time-savings, a doctor may elect to spend more time on individual surgical procedures, for example, to improve outcomes and/or to reduce doctor and/or staff stress, for example, and/or a doctor may elect to spend more time at home, or pursuing hobbies, or any number of life-balancing alternatives, for example. Additionally, for example, with such time-savings and/or improved efficiencies, a doctor may elect to perform a greater number of procedures, leading to increased income, improved finances, etc.

In one or more embodiments, VCI, including virtually-enabled on-line data entry that may be used, at least in part, to facilitate and/or support one or more virtual intake processes that may include on-line form generation, virtual consultation, informed consent origination, confirmation, and/or verification, etc., may be integrated with other systems (e.g., customer relationship management systems, etc.), for example, although, again, subject matter is not limited in scope in these respects.

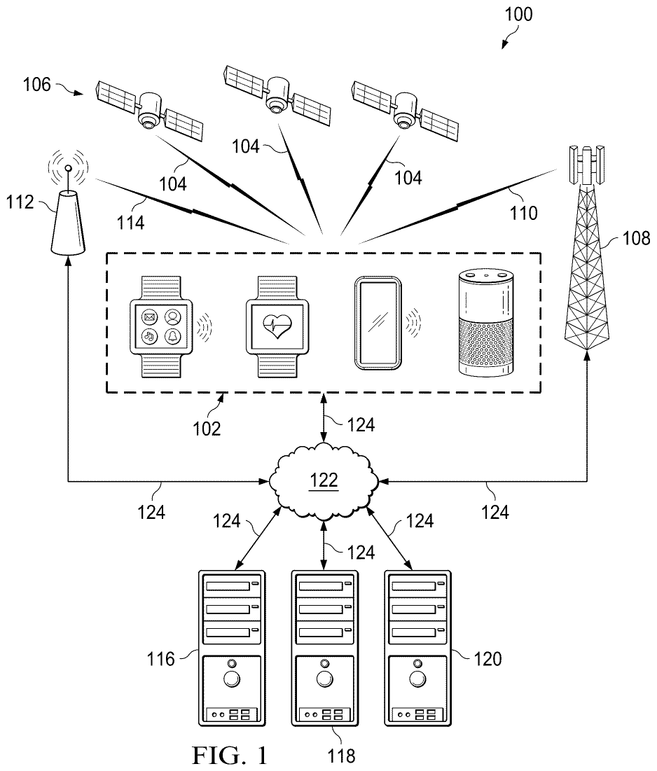

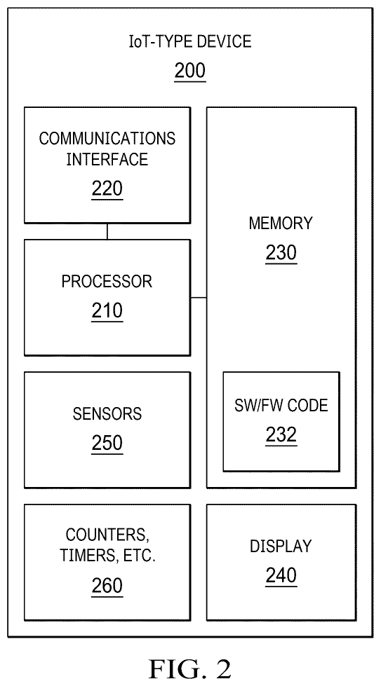

Even though a number of particular implementations are described herein, it should be noted, however, that subject matter is not limited in scope to the particular example implementations provided. Also, example implementations may utilize, at least in part, any of a wide range of computing and/or communication devices, systems, components, technologies, networks, etc. The discussion below, including aspects related to FIGS. 1 and 2, for example, describes various aspects of an example infrastructure that may facilitate and/or support, at least in part, one or more example embodiments and/or implementations discussed herein.

FIG. 1 is a schematic diagram illustrating features associated with an implementation of an example operating environment 100 capable of facilitating and/or supporting one or more operations, approaches, and/or techniques for VCI, including virtually-enabled on-line data entry that may be used, at least in part, to facilitate and/or support one or more virtual intake processes that may include on-line form generation, virtual consultation, informed consent origination, confirmation, and/or verification, etc., discussed more fully herein, for example, for distributed and/or remote computing and/or communications networks, illustrated generally herein at 102. As was indicated, one or more operations and/or techniques may, for example, be implemented. At least in part, in connection with one or more IoT-type devices, though subject matter is not so limited. Briefly, IoT is typically a system of interconnected and/or internetworked physical devices in which computing may be embedded into hardware so as to facilitate and/or support devices'abilities to acquire, collect and/or communicate content over one or more communications networks, for example, at times, without human participation and/or interaction. As mentioned, IoT-type devices may include a wide variety of stationary and/or mobile devices, such as, for example, automobile sensors, biochip transponders, heart monitoring implants, kitchen appliances, locks or like fastening devices, solar panel arrays, home gateways, smart gauges, smart telephones, cellular telephones, security cameras, wearable devices, thermostats, Global Positioning System (GPS) transceivers, personal digital assistants (PDAs), virtual assistants, laptop computers, personal entertainment systems, tablet or other personal computers (PCs), personal audio and/or video devices, personal navigation devices, mobile devices, and/or the like.

It should be appreciated that operating environment 100 is described herein as a non-limiting example that may be implemented, in whole or in part, in a context of various wired and/or wireless communications networks and/or any suitable portion and/or combination of such networks. For example, these or like networks may include one or more public networks (e.g., the Internet, the World Wide Web, etc.), private networks (e.g., intranets, etc.), wireless wide area networks (WWAN), wireless local area networks (WLAN, etc.), wireless personal area networks (WPAN), telephone networks, cable television networks, Internet access networks, fiber-optic communication networks, waveguide communication networks and/or the like. It should also be noted that claimed subject matter is not limited to a particular network and/or operating environment. Thus, for a particular implementation, one or more operations, approaches, and/or techniques for virtually-enabled on-line data entry that may be used, at least in part, to facilitate and/or support one or more virtual intake processes that may include on-line form generation, virtual consultation, informed consent origination, confirmation, and/or verification, etc., for distributed computing and/or communications networks may be performed, at least in part, in an indoor environment and/or an outdoor environment, or any combination thereof.

Thus, as illustrated, in a particular implementation, one or more computing and/or communications devices, such as IoT-type devices 102 may, for example, receive and/or acquire satellite positioning system (SPS) signals 104 from SPS satellites 106. In some instances, SPS satellites 106 may be from a single global navigation satellite system (GNSS), such as the GPS or Galileo satellite systems, for example. In other instances, SPS satellites 106 may be from multiple GNSS such as, but not limited to, GPS, Galileo, Glonass, or Beidou (Compass) satellite systems, for example. In certain implementations, SPS satellites 106 may be from any one several regional navigation satellite systems (RNSS) such as, for example, WAAS, EGNOS, QZSS, just to name a few examples.

At times, one or more IoT-type devices 102 may, for example, transmit wireless signals to and/or receive wireless signals from a suitable wireless communication network. In one example, one or more IoT-type devices 102 may communicate with a cellular communication network, such as by transmitting wireless signals to and/or receiving wireless signals from one or more wireless transmitters capable of transmitting and/or receiving wireless signals, such as a base station transceiver 108 over a wireless communication link 110, for example. Similarly, one or more IoT-type devices 102 may transmit wireless signals to and/or receive wireless signals from a local transceiver 112 over a wireless communication link 114, for example. Base station transceiver 108, local transceiver 112, etc. may be of the same or similar type, for example, and/or may represent different types of devices, such as access points, radio beacons, cellular base stations, femtocells, an access transceiver device, or the like, depending on an implementation. Similarly, local transceiver 112 may comprise, for example, a wireless transmitter and/or receiver capable of transmitting and/or receiving wireless signals. For example, at times, wireless transceiver 112 may be capable of transmitting and/or receiving wireless signals from one or more other terrestrial transmitters and/or receivers.

In a particular implementation, local transceiver 112 may, for example, be capable of communicating with one or more IoT-type devices 102 at a shorter range over wireless communication link 114 than at a range established via base station transceiver 108 over wireless communication link 110. For example, local transceiver 112 may be positioned in an indoor or like environment and/or may provide access to a WLAN (e.g., IEEE Std. 802.11 network, etc.) and/or WPAN (e.g., Bluetooth® network, etc.). In another example implementation, local transceiver 112 may comprise a femtocell and/or picocell capable of facilitating communication via link 114 according to an applicable cellular or like wireless communication protocol. Again, it should be understood that these are merely examples of networks that may communicate with one or more IoT-type devices 102 over a wireless link, and claimed subject matter is not limited in this respect. For example, in some instances, operating environment 100 may include a larger number of base station transceivers 108, local transceivers 112, networks, terrestrial transmitters and/or receivers, etc.

In an implementation, one or more IoT-type devices 102, base station transceiver 108, local transceiver 112, etc. may, for example, communicate with one or more servers, referenced herein at 116, 118, and 120, over a network 122, such as via one or more communication links 124. It should be noted that, depending on an implementation, one or more servers 116, 118, and/or 120 may be part of a centralized network, a decentralized network, or any combination thereof. Thus, even though terms like “server” or “servers’ are used herein, such as for ease of discussion, it should be appreciated that these or like aspects may include and/or be part of one or more centralized and/or decentralized networks. Thus, as indicated, network 122 may comprise, for example, a centralized network, a decentralized network, or any combination thereof, and may include any number and/or combination of wired and/or wireless communication links. In a particular implementation, network 122 may comprise, for example, Internet Protocol (IP)-type infrastructure capable of facilitating or supporting communication between one or more IoT-type devices 102 and one or more servers 116, 118, 120, etc. via local transceiver 112, base station transceiver 108, directly, etc. In another implementation, network 122 may comprise, for example cellular communication network infrastructure, such as a base station controller and/or master switching center to facilitate and/or support mobile cellular communication with one or more IoT-type devices 102. Servers 116, 118 and/or 120 may comprise any suitable servers or combination thereof capable of facilitating or supporting one or more operations and/or techniques discussed herein. For example, servers 116, 118 and/or 120 may comprise one or more update servers, back-end servers, management servers, archive servers, location servers, positioning assistance servers, navigation servers, map servers, crowdsourcing servers, network-related servers, or the like.

Even though a certain number of computing platforms and/or devices are illustrated herein, any number of suitable computing platforms and/or devices may be implemented to facilitate and/or support one or more techniques and/or processes associated with operating environment 100. For example, at times, network 122 may be coupled to one or more wired and/or wireless communication networks (e.g., WLAN, etc.) so as to enhance a coverage area for communications with one or more IoT-type devices 102, one or more base station transceivers 108, local transceiver 112, servers 116, 118, 120, or the like. In some instances, network 122 may facilitate and/or support femtocell-based operative regions of coverage, for example. Again, these are merely example implementations, and claimed subject matter is not limited in this regard.

In this context, “IoT-type devices” refer to one or more electronic and/or computing devices capable of leveraging existing Internet or like infrastructure as part of the so-called “Internet of Things” or IoT, such as via a variety of applicable protocols, domains, applications, etc. As was indicated, the IoT is typically a system of interconnected and/or internetworked physical devices in which computing may be embedded into hardware so as to facilitate and/or support devices'ability to acquire, collect, and/or communicate content over one or more communications networks, for example, at times, without human participation and/or interaction. IoT-type devices 102, for example, may include a wide variety of stationary and/or mobile devices, such as, for example, automobile sensors, biochip transponders, heart monitoring implants, kitchen appliances, locks or like fastening devices, solar panel arrays, home gateways, smart gauges, smart telephones, cellular telephones, security cameras, wearable devices, thermostats, GPS transceivers, PDAs, virtual assistants, laptop computers, personal entertainment systems, tablet PCs, PCs, personal audio or video devices, personal navigation devices, mobile devices, stationary devices, and/or the like, to name a few non-limiting examples. Typically, in this context, a “mobile device” refers to an electronic and/or computing device that may from time to time have a position or location that changes, and/or a “stationary device” refers to an electronic and/or computing device that may have a position or location that generally does not change. In some instances, IoT-type devices, such as IoT-type devices 102, may be capable of being identified, such as uniquely, via an assigned IP address (e.g., static, dynamic, etc.), as one particular example, and/or having an ability to communicate, such as receive and/or transmit electronic content, for example, over one or more wired and/or wireless communications networks.

It may again be noted that the example infrastructure discussed above, along with additional systems, apparatuses, processes, etc., discussed herein, may be directed, at least in part, to supporting virtual consultation operations, approaches, and/or techniques, such as those discussed herein, for example. In one or more embodiments, an example infrastructure described in connection with FIG. 1 and/or FIG. 2, for example, may provide a platform to support example functionalities described herein, including operations, approaches, and/or techniques pertaining to machine-learning/AI-based (at least in part) virtual consultation, such as may include virtually-enabled on-line data entry that may be used, at least in part, to facilitate and/or support one or more virtual intake processes that may include on-line form generation, virtual consultation, informed consent origination, confirmation, and/or verification, etc., for example. It may also be appreciated that one or more software and/or firmware agents implemented to facilitate example functionalities described herein may be partitioned between administrator computing devices, such as servers 120, for example, and any number of remote and/or mobile devices, such as IoT-type devices 102, for example, that may be associated with one or more patients, doctors, staff members, etc.

FIG. 2 is an illustration of an embodiment 200 of an example particular IoT-type device. Of course, subject matter is not limited in scope to the particular configurations and/or arrangements of components depicted and/or described for example devices mentioned herein. In an embodiment, an IoT-type device, such as 200, may comprise one or more processors, such as processor 210, and/or may comprise one or more communications interfaces, such as communications interface 220. In an embodiment, one or more communications interfaces, such as communications interface 220, may enable wireless and/or wired communications between an electronic device, such as an IoT-type device 200, and one or more other computing devices. In an embodiment, wireless and/or wired communications may occur substantially in accordance any of a wide range of communication protocols, such as those known and/or mentioned herein, for example, and/or developed in the future.

In a particular implementation, an IoT-type device, such as IoT-type device 200, may include a memory, such as memory 230. In a particular implementation, memory 230 may comprise a non-volatile memory, for example. Further, in a particular implementation, a memory, such as memory 230, may have stored therein executable instructions, such as for one or more operating systems, communications protocols, and/or applications, for example. A memory, such as 230, may further store particular instructions, such as software and/or firmware code 232, that may be updated via one or more example implementations and/or embodiments described herein. Further, in a particular implementation, an IoT-type device, such as IoT-type device 200, may comprise a display, such as display 240, and/or one or more sensors, such as one or more sensors 250. As utilized herein, “sensors” and/or the like refer to a device and/or component that may respond to physical stimulus, such as, for example, heat, light, sound pressure, magnetism, particular motions, etc., and/or that may generate one or more signals and/or states in response to physical stimulus. Example sensors may include, but are not limited to, one or more accelerometers, gyroscopes, thermometers, magnetometers, barometers, light sensors, proximity sensors, hear-rate monitors, perspiration sensors, hydration sensors, breath sensors, cameras, microphones, etc., and/or any combination thereof. It may be noted that IoT-type devices, such as device 200, may include one or more cameras to facilitate video recording, for example.

In particular implementations, IoT-type device 200 may include one or more timers and/or counters and/or like circuits, such as circuitry 260, for example. In an embodiment, one or more timers and/or counters and/or the like may track one or more aspects of device performance and/or operation. For example, timers, counters, and/or other like circuits may be utilized, at least in part, by IoT-type device 200 to determine measures of fitness, for example, and/or to otherwise generate feedback content related to testing results, in particular implementations.

Although FIG. 2 depicts a particular example implementation of an IoT-type device, such as IoT-type device 200, other embodiments may include other types of electronic and/or computing devices. Example types of electronic and/or computing devices may include, for example, any of a wide range of digital electronic devices, including, but not limited to, cellular telephones (e.g., smartphones, etc.), tablet devices, desktop and/or notebook computers, virtual and/or augmented reality devices, high-definition televisions, digital video players and/or recorders, game consoles, satellite television receivers, wearable devices, personal digital assistants, mobile audio and/or video playback and/or recording devices, streaming devices, or any combination of the foregoing. Of course, subject matter is not limited in scope in these respects.

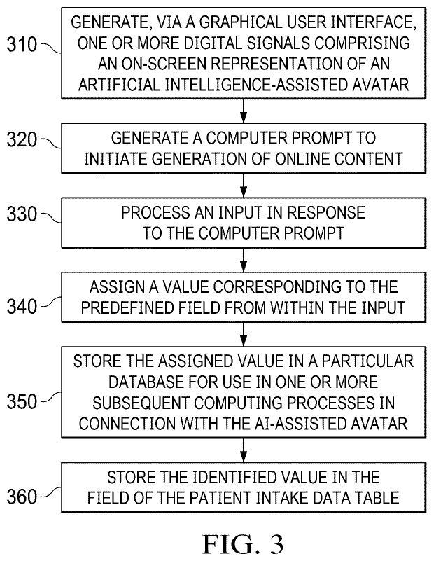

FIG. 3 is an example flow diagram illustrating an example method for virtually-enabled online content generation in accordance with one or more embodiments. At stage 310, a computing device can generate, via a GUI, one or more digital signals comprising an on-screen representation of an artificial intelligence (“AI”)-assisted avatar. A computing device can be any processor-based device, including, for example, a desktop computer, laptop, tablet, smartphone, server, kiosk terminal, or network-connected workstation, such as a workstation located within a dental or oral surgery office. In some implementations, a computing device can be a local client device executing a browser-based or native graphical application. In some implementations, a computing device can be a processor-based device included in a VCI, such as, for example, the VCI discussed previously herein. In some implementations, the GUI can be delivered from a remote server through a web interface, with rendering occurring on a client device while primary processing occurs on a cloud-based backend system. The computing device can include a display, microphone, speaker, network interface, and one or more processors executing program instructions stored in memory. The GUI can occupy all or a portion of the display and may include various visual components such as form fields, buttons, progress indicators, or conversation transcripts.

The GUI can render an avatar configured to visually represent an AI-assisted virtual intake assistant. The avatar can be implemented as a two-dimensional animation, a three-dimensional model, a hybrid graphic element capable of simulating facial movement and expressions, etc. The avatar can be displayed within a viewport region of the GUI and can exhibit synchronized motion or lip movement corresponding to generated audio output. The GUI can further include additional visual features such as subtitles, a text transcript of the conversation, and one or more controls allowing the user to adjust playback volume, repeat a question, or transition to manual data entry. In certain configurations, the GUI can highlight the current data field associated with the avatar's prompt, for example, by emphasizing the corresponding input box within a patient intake form.

The avatar displayed within the GUI can be backed by a backend VCI service that manages dialogue state and generates avatar behaviors. As used herein, the term “backend service” can refer to a collection of server-side software components, processes, and data management systems that support the operation of the application and avatar interface. These services can handle the logic, data processing, and inter-service communication necessary for the system to function reliably, securely, and in real time. The backend service can execute one or more artificial intelligence models, such as a conversational dialogue model or a natural language generation (NLG) model, that determine the avatar's utterances, tone, and timing based on the current intake context. The backend service can also provide data synchronization between the avatar's dialogue flow and the patient intake data table stored in a database. In some implementations, the avatar's visual and auditory behavior can be controlled through metadata transmitted from the backend to the client device, such as phoneme timing for lip synchronization, gesture triggers, or expression parameters. The backend service can further maintain session data that identifies the user, tracks progress through the intake sequence, and stores audit information used to reconstruct or review prior intake interactions.

At stage 320, the computing device can generate a computer prompt to initiate generation of online content. The computer prompt can serve as a trigger for producing or retrieving dynamic digital content that is to be presented to a user through a GUI. The online content can include visual, auditory, or interactive elements—such as digital forms, dialogue windows, or avatar-driven interactions—that support the intake or consultation workflow. In some embodiments, the online content can be partitioned on a display screen of a client device according to one or more predefined portions. Each predefined portion can correspond to a functional region of the interface, such as an area dedicated to an AI-assisted avatar, another area reserved for user input or form content, and one or more additional areas for contextual information, controls, or navigation elements. The predefined portions can be defined by layout parameters stored in a configuration file or database and can be dynamically adjusted based on user activity, device characteristics, or workflow progression. For example, while an avatar is actively engaging with the user, the portion allocated to the avatar may expand while other interface sections are temporarily minimized to maintain focus on the conversation.

The computer prompt that initiates the content generation can comprise at least one of the following: an audio prompt, an on-screen prompt, a haptic prompt, or any combination thereof. The audio prompt can include synthesized speech generated by a text-to-speech model that verbally guides the user or announces the availability of new content. The on-screen prompt can include visual indicators such as flashing icons, highlighted sections, animated text, or graphical transitions that draw attention to the next interaction or content segment. The haptic prompt can include tactile feedback—such as vibration or pressure signals—generated by the client device to alert the user that a new interaction is ready or that input is required. In some embodiments, multiple prompt types can be used concurrently to reinforce engagement and accessibility; for instance, an audio prompt may be accompanied by a subtle vibration or visual cue. Backend services can coordinate the timing and delivery of these prompts, ensuring that the appropriate content and layout are retrieved from storage, formatted, and rendered in the correct predefined portions of the display. This architecture allows the system to maintain a responsive, multimodal user experience that adapts to both the user's interaction style and the operational context of the digital intake process.

At stage 330, the computing device can process an input in response to the computer prompt. The input can include any form of user-provided interaction that responds to the prompt generated at stage 320. Examples of such input can include textual input entered through a keyboard or touchscreen, speech input captured by a microphone, a gesture or selection made using a cursor or touch interface, or a combination thereof. The computing device can monitor one or more input channels—such as an audio input interface, a graphical user interface event handler, or a sensor array—to detect and register the user's response. Once the input is detected, the computing device can initiate a corresponding data processing operation, such as parsing the input, validating the format, or associating it with a predefined field or command.

In some embodiments, the input can be multimodal, meaning that the user may provide both voice and manual input in response to the same prompt. For example, a user may verbally state an answer while simultaneously confirming or editing the text using an on-screen control. The computing device can apply input prioritization logic or fusion algorithms to combine or reconcile multiple forms of input. If the input is a spoken response, the computing device can route the audio data through a speech processing pipeline, converting the speech to text and analyzing the text using NLP models to determine the user's intent. If the input is a manual interaction, such as selecting a button, typing text, or uploading a file, the computing device can process the event using a user interface controller that maps the input to the corresponding action or data field within the graphical interface.

On the backend, an input management service can coordinate the handling, interpretation, and validation of user inputs. The service can log all received inputs, associate them with session identifiers, and route them to appropriate subsystems—such as a data storage module, workflow engine, or AI dialogue manager—for further processing. The backend services can also perform error detection and recovery functions, such as prompting the user to repeat or correct incomplete responses. In some implementations, the AI-assisted avatar can provide real-time feedback as the computing device processes the input, confirming receipt of valid information or providing guidance if the input does not meet expected criteria. This stage thus enables seamless interaction between the user and the system, ensuring that each response to a computer prompt is accurately captured, interpreted, and integrated into the ongoing digital workflow.

At stage 340, the computing device can assign a value corresponding to the predefined field from within the input. For example, the computing device can analyze the content of the input to determine which portion of the input corresponds to a specific data field defined in a patient intake data table or other structured dataset. The predefined field can represent a specific category of information, such as a patient's name, date of birth, insurance provider, or contact number. The computing device can identify this field based on contextual information from the workflow, metadata associated with the computer prompt, or patterns detected within the input itself.

In some embodiments, the computing device can implement parsing logic or NLP techniques to extract relevant values from unstructured or semi-structured input. For instance, if the input includes a full sentence such as “My date of birth is June twenty-first, nineteen eighty,” the computing device can isolate and normalize the date value “Jun. 21, 1980” and assign it to the predefined “Date of Birth” field. If the input includes multiple potential data elements, the computing device can apply field-specific rules or confidence scoring to determine which extracted value best matches the intended field. The assignment operation can therefore include identifying a substring, token sequence, or numerical pattern that satisfies the criteria associated with the target field and binding that value to a data object within the intake record.

On the backend, a field-mapping or schema management service can coordinate how values are assigned to specific data structures. Each field in the data table can be associated with a field identifier, data type constraint, and validation rule. If a value is identified within the input, the backend service can verify its format and data type before committing it to storage. For example, numeric-only fields such as medical record numbers can be validated for character length, while date fields can be standardized to a consistent format. The computing device can also log each value assignment operation with a timestamp and session identifier, enabling auditability and error tracking. In some implementations, the AI-assisted avatar can provide immediate confirmation or correction prompts in real time, such as verbally confirming, “I've recorded your date of birth as Jun. 21, 1980—shall I continue?” before finalizing the assignment.

At stage 350, the computing device can store the assigned value in a particular database for use in one or more subsequent computing processes in connection with the AI-assisted avatar. For example, the computing device can transmit the value—along with associated metadata such as the session identifier, timestamp, field identifier, etc.—to a backend database or data storage system. The database can include, for example, a structured or semi-structured data repository such as a relational database, document-oriented database, or cloud-based data store. The database may be configured to organize patient intake information in association with user profiles, session data, and workflow context, allowing the stored values to be accessed and used across multiple components of the system.

In some embodiments, the computing device can employ a data persistence or synchronization service to ensure reliable storage and retrieval of the assigned value. The persistence layer can handle data formatting, indexing, and encryption prior to storage, and can verify successful write operations through confirmation signals or integrity checks. The VCI can also maintain versioning records for each stored field, allowing changes to be tracked and audited over time. Once stored, the assigned value can be linked to the specific user or patient record, enabling subsequent computing processes to reference or update that information as needed.

The stored value can be utilized in various downstream computing operations involving the AI-assisted avatar and other system components. For example, the avatar may retrieve stored data during future interactions to provide personalized prompts, confirm previously entered information, or pre-populate related form fields. The backend may also employ the stored values in analytics, scheduling, insurance verification, or documentation generation workflows. In certain implementations, the storage operation can trigger automated functions, such as notifying a backend service that a particular data field has been completed, updating workflow progress metrics, or initiating a new sequence of avatar-driven dialogue. Through this process, the VCI platform ensures that each user-provided and system-validated value becomes an accessible, persistent element of the digital intake dataset, enabling continued interaction and intelligent behavior of the AI-assisted avatar across sessions and related computing processes.

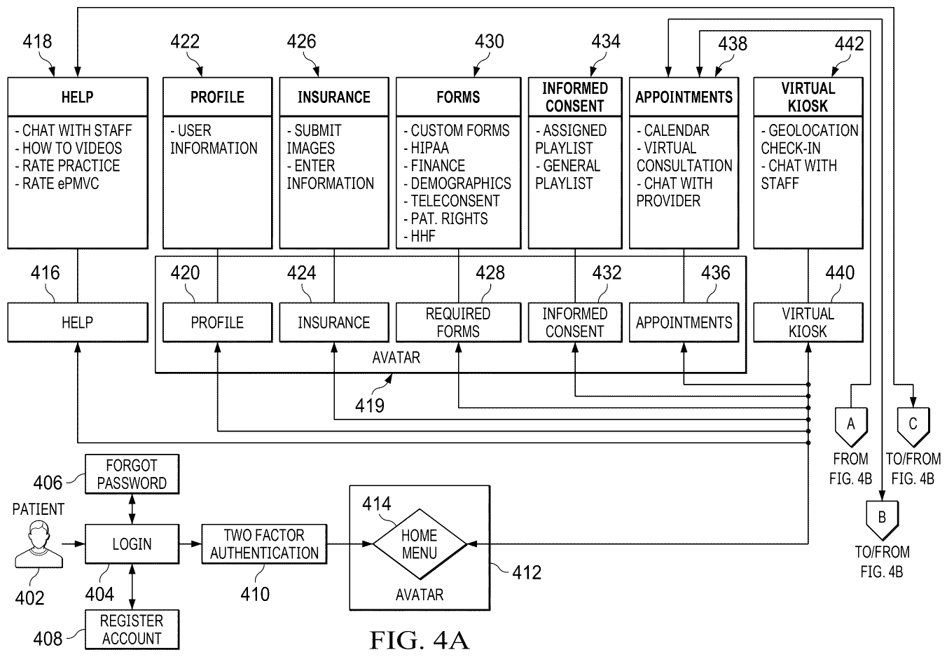

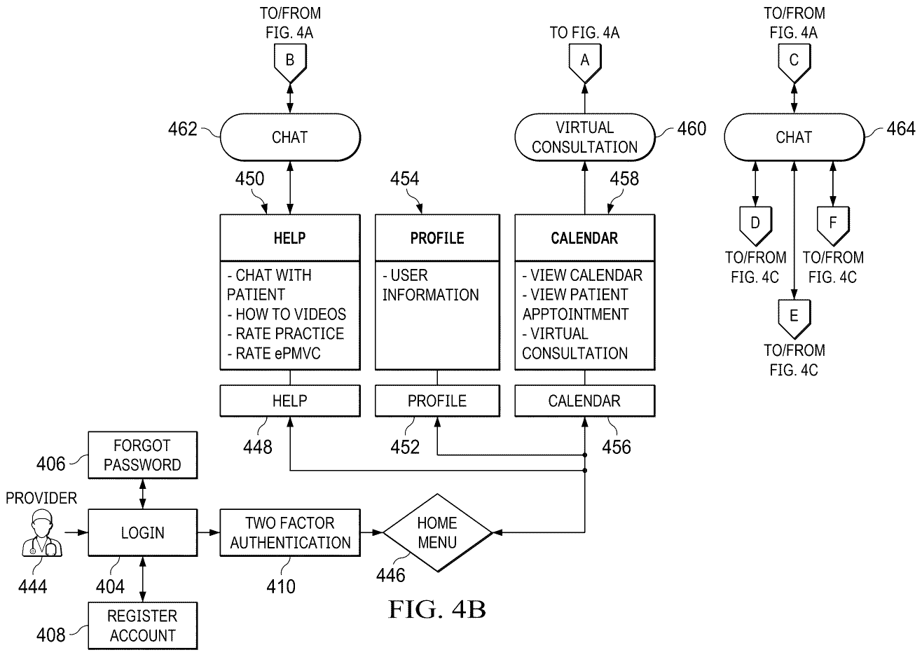

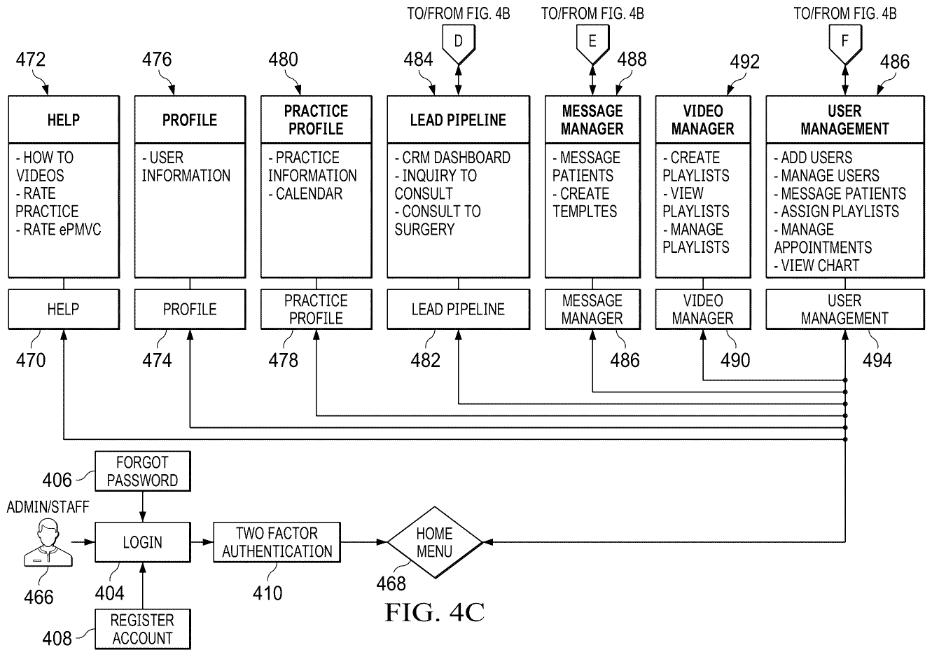

FIGS. 4A-4C are a flow diagram depicting example processes for virtual consultation for medical and/or other professionals in accordance with one or more embodiments. FIG. 4A shows an initial authentication and navigation sequence executed prior to initiating the intake workflow.

A patient user 402 can represent any individual seeking dental, oral surgery, or other healthcare services facilitated by the digital intake system. The patient user 402 can access the platform through a computing device such as a smartphone, tablet, laptop, desktop workstation, or a kiosk terminal located in a clinic or reception area. In some embodiments, the patient user 402 may be a first-time visitor registering for an appointment, an existing patient updating their information, or a returning user preparing for a scheduled procedure. The client device used by the patient user 402 can execute a native application or a web-based portal and can communicate with one or more backend services over a secure network connection. These backend services can include authentication modules, database servers, user profile repositories, and AI processing systems configured to support avatar functionality and data exchange.

Upon initiating the application, the patient user 402 can be presented with a login screen 404, which can include user interface fields for submitting authentication credentials such as a username, email address, phone number, or password. The login screen 404 can further include a forgot password link 406, which, if selected, can trigger a password recovery process involving email or SMS-based verification, and a register account link 408, which allows a new user to create an account. During registration, backend services can generate a unique patient identifier, create a new user profile, and securely store credentials in an encrypted authentication database.

After the user enters valid credentials, the platform can proceed to a two-factor authentication stage 410, where additional verification is performed to enhance security. The backend authentication module can generate a one-time passcode (OTP) and transmit it to a registered communication channel such as the user's mobile device, email, or an authentication app. Upon receiving the OTP, the patient user 402 can input the code into the client interface, and the backend can validate the code against a temporary session token. Successful verification results in the creation of a secure session and the issuance of an access token that governs authenticated interaction with the platform.

Once authentication is complete, the platform can transition to a patient home menu 414, where an AI-assisted avatar 412 can be presented to the patient user 402. The avatar can be displayed as a two-or three-dimensional graphical representation capable of speech synthesis and natural language interaction. Backend components such as a dialogue management engine, natural language understanding (NLU) model, and context-tracking module can coordinate the avatar's behavior and dialogue flow. The patient home menu 414 can serve as a central navigation hub, allowing the patient user 402 to select from a range of menu options associated with the intake process.

The patient home menu 414 can include multiple selectable options, each linked to a corresponding functional module. In some examples, the options can include a profile section 420 for managing personal data, an insurance section 424 for uploading or verifying insurance information, a required forms section 428 for completing digital intake paperwork, an informed consent section 432 for reviewing and electronically signing consent documents, and an appointments section 416 for scheduling or reviewing upcoming visits. Certain menu items, such as a help section 416 and a virtual kiosk section 440, may operate independently of the avatar 412 but remain accessible through the same interface. The avatar 412 can assist with some or all of these sections by guiding the user through data entry, explaining form content, or answering natural language queries.

Selection of a menu option, or a voice indication of a menu choice provided to the avatar 412, can cause a corresponding submenu to be displayed. Each submenu can provide additional functionality and data entry capabilities. For example, a help submenu 418 can include options such as initiating a chat with clinic staff, viewing instructional videos, submitting practice ratings, or providing an electronic patient management and virtual care score. A profile submenu 422 can display user-specific demographic data, allowing the patient user 402 to review or update their contact information, address, or emergency contact details. An insurance submenu 426 can provide fields for entering policy information and functionality for uploading images, such as photographs of an insurance card, which can be processed by backend OCR (optical character recognition) services.

A required forms submenu 430 can present various digital documents that the patient user 402 may be required to complete, such as HIPAA authorization forms, financial agreements, demographic questionnaires, teleconsent documents, patient rights disclosures, and health history forms (HHF). These forms can be generated dynamically by backend document services based on the patient's appointment type, procedure, or insurance provider. An appointments submenu 438 can include features such as a calendar view of scheduled visits, a virtual consultation interface, and a secure messaging channel for communicating directly with healthcare providers. Finally, a virtual kiosk submenu 442 can include features such as geolocation-based check-in, enabling the patient user 402 to automatically register their arrival at a physical clinic location, and a real-time chat interface for interacting with administrative staff.

On the backend, each menu and submenu can be tied to corresponding service modules that manage data storage, retrieval, and synchronization. For instance, profile updates can be written to a patient record database, insurance data can be transmitted to a verification API, and completed forms can be stored in a secure EHR repository. All user actions can be logged and time-stamped, allowing for auditability and traceability throughout the intake workflow. These backend operations enable the platform to present a seamless, conversational user experience through the avatar interface while maintaining structured, secure handling of sensitive healthcare data.

FIG. 4B illustrates an example workflow for provider-side users within the AI-assisted intake application. A provider user 444 can represent any healthcare professional, such as a dentist, oral surgeon, hygienist, treatment coordinator, or administrative staff member, who interacts with the platform to manage patient intake, review schedules, conduct consultations, or communicate with patients. Provider users 444 can access the platform using a computing device such as a desktop workstation, tablet, laptop, or mobile device, either from within a clinical setting or remotely via a secure connection. Like patient users, provider users 444 can authenticate with the platform through a login screen, password verification, and optional two-factor authentication processes, ensuring secure access to patient data and protected health information (PHI).

Once authenticated, the provider user 444 can be presented with a provider home menu 446, which serves as the central navigation interface for provider-side functionality. In contrast to the patient-facing interface, the provider interface can omit the AI-assisted avatar and instead present a streamlined, utilitarian menu designed for clinical and administrative workflows. The provider home menu 446 can include a plurality of menu options such as a help section 448, a profile section 452, and a calendar section 456. Each section can be associated with one or more backend services, including scheduling databases, patient information repositories, messaging systems, and communication APIs, to support real-time interaction and data synchronization between the provider's device and the platform's core infrastructure.

Selection of the help submenu 450 can present several tools to support provider interactions and system navigation. These tools can include a real-time messaging feature that enables the provider user 444 to initiate and manage chat sessions with patient users 402, video tutorials and “how-to” guides that explain platform features, and interfaces for submitting feedback such as practice ratings or application ratings. Communication initiated through the help submenu 450 can be routed through a backend messaging service that maintains secure message delivery, message history, and session metadata.

The profile submenu 454 can allow the provider user 444 to add, edit, or update their professional information stored within the platform. Examples of such data can include contact details, areas of specialization, professional credentials, and office hours. Any changes submitted through the profile submenu 454 can trigger a write operation to a secure provider database, where the platform can validate and store the updated records. In some cases, profile data may also synchronize with third-party systems, such as EHR platforms or scheduling tools, through secure APIs.

The calendar submenu 458 can provide the provider user 444 with a comprehensive view of their clinical schedule. This can include daily, weekly, or monthly appointment views, as well as filtering tools for viewing appointments by patient, procedure type, or location. The calendar interface can be dynamically updated by a backend scheduling engine that aggregates appointment data from multiple sources, including patient-initiated bookings and administrative scheduling systems. From this interface, the provider user 444 can select individual patient appointments to view additional details, such as appointment type, patient history, or required intake forms.

The platform can include a feature that facilitates virtual consultation 460 for provider and patient. A virtual consultation 460 can be established if both the provider user 444 and the patient user 402 select the appropriate option within their respective interfaces. For the patient user 402, this option may be accessed through the appointments submenu 438 described previously, while for the provider user 444, it may be accessed through the calendar submenu 458. Once initiated, the platform can coordinate a secure, real-time video communication session between the patient and provider. The backend can employ video conferencing APIs, encryption protocols, and session management services to establish and maintain the connection. The virtual consultation 460 can allow for patient evaluation, treatment planning, and pre-procedure discussions without requiring in-person visits.

In addition to video consultations, the platform can support asynchronous or real-time text-based communication through a chat interface 462. This messaging capability allows provider users 444 and patient users 402 to exchange messages within the platform. For patient users, the chat interface 462 can be accessed through the appointments submenu 438 or the help submenu 418, while provider users 444 can access it through the help submenu 450. The backend messaging infrastructure can support message encryption, delivery confirmation, timestamping, and automatic archiving to comply with privacy regulations and record-keeping requirements. This messaging channel can be used for follow-up instructions, clarification of intake information, pre-appointment guidance, or post-procedure communication.

Overall, the workflow depicted in FIG. 4B illustrates how provider users 444 interact with the platform to manage patient engagement, maintain clinical schedules, and conduct consultations, all while leveraging a secure backend infrastructure that facilitates real-time data synchronization, communication, and collaboration. Together with the patient-facing workflow shown in FIG. 4A, the provider workflow enables coordinated, end-to-end digital interaction between patients and providers within the same integrated platform.

FIG. 4C illustrates an example workflow for admin-side users within the AI-assisted intake application. An admin user 466 can represent a clinic administrator, office manager, operations coordinator, or other authorized personnel responsible for overseeing system operations, managing users, and configuring the platform to support patient care workflows. Like patient and provider users, the admin user 466 can authenticate with the platform through a secure login process, which may include credential verification, two-factor authentication, and session token issuance. Upon successful authentication, the admin user 466 can be presented with an admin home menu 468, which serves as a central dashboard for managing a wide range of administrative tasks and system configurations.

The admin home menu 468 can provide access to several key functional areas, including a help section 470, a profile section 474, a practice profile section 478, a lead pipeline section 482, a message manager section 486, a video manager section 490, and a user management section 494. Each of these sections can correspond to distinct backend modules that manage associated data and workflows. The backend infrastructure can include application servers, databases, content delivery networks, and messaging services that handle data retrieval, synchronization, and execution of administrative commands in real time.

The help submenu 472 can provide resources designed to support the administrative use of the platform. These resources can include instructional “how-to” videos, reference guides, and performance feedback tools such as practice ratings and virtual care ratings. Selections from this submenu can trigger backend requests for media assets or analytics data, which may be streamed or retrieved from a centralized knowledge repository.

The profile submenu 476 allows the admin user 466 to view, add, or update their own user information. Such data may include contact details, role-specific permissions, or notification preferences. Changes made in this section can be validated by backend services and written to a secure user profile database, ensuring that administrative records remain current and compliant with organizational policies.

The practice profile submenu 480 enables the admin user 466 to view and modify practice-related information, such as the clinic's name, contact information, address, office hours, staff assignments, and service offerings. The submenu can also include controls for viewing and managing the practice's master calendar, including scheduling availability, appointment types, and operational blocks. Updates made in this section can propagate across the platform, synchronizing with patient-and provider-facing interfaces to ensure consistent scheduling and availability data.

The lead pipeline submenu 484 can include tools for managing patient acquisition and conversion workflows. A Customer Relationship Management (CRM) dashboard can provide a real-time overview of patient inquiries, lead status, and conversion metrics. Additional features, such as an inquiry-to-consult module and a consult-to-surgery module, can automate the tracking of patients as they move through different stages of the treatment pipeline. Backend analytics services can process lead data, generate conversion metrics, and surface actionable insights for the administrative team, enabling more efficient patient outreach and follow-up.

In one or more embodiments, selection of ‘Inquiry to Consult’ may result in display of a pie chart representing a distribution of patients across different workflow stages, for example. In one or more embodiments, a pie chart may categorize patients into the following example workflow stages: Insurance Info, Required Forms, Informed Consent and/or Virtual Consultation, for example. In one or more embodiments, each piece of a pie chart may be segmented based at least in part on different percentages of active patients in each current workflow stage.

In one or more embodiments, VCI may update pie chart in real-time or near real-time as patients complete individual stages of a workflow, for example. In one or more embodiments, content (e.g., data, etc.) for a pie chart may be sourced from a patient management system database, for example. In one or more embodiments, in a “Users” section of a database, for example, fields may be added. For example, a “currentStep” field may designate a specific workflow stage for each patient and a “currentStepTimestamp” field may record date and/or time if the patient reaches or finishes individual aspects of a workflow, in one or more embodiments.

In one or more embodiments, individual pieces of a pie chart may have a total number of patients that are on a stage of the workflow, for example. Each tile may show a number of patients in a workflow stage at the left side of the tile, for example. In one or more embodiments, below the chart, a VCI system may display tiles corresponding to each workflow stage, for example. In one or more embodiments, users may be able to click on each tile to access a patient list in that specific workflow stage, for example.

The message manager submenu 488 provides communication management capabilities, allowing the admin user 466 to compose, send, and track messages sent to patient users 402 or provider users 444. The admin user 466 can also create and store message templates for common communication scenarios, such as appointment reminders, payment notifications, or pre-procedure instructions. Messages can be delivered via multiple channels—including in-app messaging, email, or SMS—through a backend messaging service that handles delivery status, encryption, and logging.

The video manager submenu 492 allows the admin user 466 to create, upload, organize, and manage video content, including patient education materials, post-operative care instructions, promotional content, and instructional guides. Admin users can group videos into playlists and assign them to specific patient categories, procedures, or care pathways. The backend system can handle video encoding, storage, access control, and streaming, ensuring that content is delivered efficiently and securely to patient devices.

The user management submenu 494 provides robust administrative control over the platform's user base. The admin user 466 can add or remove users, manage user roles and permissions, and initiate communication with patients directly from this interface. Additional capabilities can include assigning video playlists, managing appointments, viewing patient charts, and overseeing usage analytics. The backend system can enforce access control policies, maintain audit trails of administrative actions, and ensure compliance with privacy and data protection regulations.

A chat interface 464 can allow the admin user 466 to communicate directly with patient users 402. This functionality can support real-time assistance, follow-ups on incomplete forms, reminders for upcoming appointments, or responses to patient inquiries. Messages sent through this interface can be routed through the same secure messaging infrastructure used by patient and provider communications, ensuring consistency and data integrity across all communication channels.

Overall, the workflow illustrated in FIG. 4C provides administrative users with comprehensive tools for managing the operational, clinical, and communication aspects of the AI-assisted intake platform. By integrating these functions into a single unified interface, the platform allows administrators to coordinate patient engagement, streamline clinic operations, monitor performance metrics, and maintain high-quality digital interactions across patient, provider, and administrative roles.

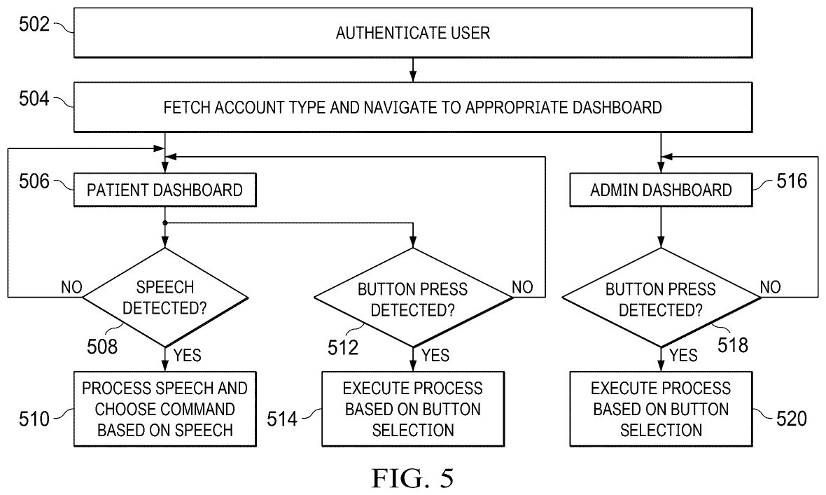

FIG. 5 illustrates an example flowchart depicting the logic by which the AI-assisted intake platform interacts with patient and administrative users, processes input, and executes corresponding actions. This workflow demonstrates how the platform's backend services coordinate authentication, account classification, dashboard rendering, input interpretation, and process execution across different user roles.

At stage 502, the platform authenticates a user attempting to access the platform. This authentication process can involve verifying user credentials (e.g., username, password, token, etc.) submitted through a login interface, and may also include a multi-factor authentication (MFA) sequence to enhance security. During this stage, the platform's backend authentication module can interface with a secure identity provider (IdP) or authentication service, which validates credentials against a user account database. Authentication tokens or session keys may be generated and assigned upon successful login, allowing secure, stateful interaction throughout the session. The authentication module may also log metadata such as device identifiers, geolocation information, and IP addresses for security auditing and anomaly detection.

At stage 504, once authentication is complete, the platform can fetch the user's account type from a centralized user management service or role-based access control (RBAC) database. The backend system queries stored account attributes, which may include user role (e.g., patient, provider, admin, etc.), associated permissions, and linked data objects. Based on this information, the platform dynamically routes the user to the appropriate dashboard. For example, a patient user is directed to a patient dashboard 506, which includes patient-facing features such as the AI-assisted avatar and voice interaction capabilities, whereas an administrative user is directed to an admin dashboard 516, which presents management and operational tools without the avatar interface. This routing process can include backend checks for feature entitlements, subscription tiers, or organizational configurations to determine which dashboard modules are available to the user.

Once the patient dashboard 506 is rendered, the platform continuously monitors for user input events through multiple modalities. At stage 508, the platform detects whether speech input is received. Audio input monitoring can involve real-time audio streaming from a microphone interface, processed by a voice activity detection (VAD) engine to distinguish speech from background noise. If speech is detected, the audio data is passed to a backend ASR pipeline that converts speech to text. The resulting text is then analyzed by an NLU model at stage 510, which classifies user intent and extracts key parameters. The platform's command interpretation service maps the processed input to a specific action or workflow, such as navigating to a submenu, submitting a data field, retrieving appointment information, or initiating a virtual consultation. Command selection can involve rule-based decision trees, machine learning-based intent classifiers, or hybrid approaches that leverage both context and user history to infer the most relevant action.

If no speech input is detected, the platform can monitor for other forms of interaction. At stage 512, the platform determines whether a button selection or graphical input event has occurred. Button selections can include clicks, taps, or other GUI interactions performed by the patient user. Upon detection, the platform's frontend client transmits an event payload to the backend interaction manager, which validates the event, retrieves any required data, and dispatches a corresponding operation at stage 514. For example, a button selection may trigger the retrieval of insurance information, submission of a completed intake form, or navigation to a scheduling interface. All actions executed through this pathway are logged and linked to the user session for audit and compliance purposes.

If an administrative user is directed to the admin dashboard 516, the platform loads a separate interface optimized for practice management, scheduling, messaging, and analytics. Similar to the patient interface, the platform monitors user actions and event streams. At stage 518, the backend event listener checks for button selections or input events within the administrative UI. If a button selection is detected, the event is transmitted to the backend execution layer at stage 520, where the platform initiates the corresponding process. Such processes can include retrieving patient data, generating performance reports, updating practice information, creating video playlists, sending broadcast messages, or managing user permissions.

The backend logic supporting administrative workflows can involve multiple subsystems. For example, scheduling operations may interface with a calendar service and a database of appointment records, messaging actions may pass through a communication gateway with encryption and delivery tracking, and lead management functions may interact with a CRM database to update pipeline metrics. The backend system can further enforce access controls, ensuring that administrative actions conform to organizational policies and that sensitive patient data remains protected under applicable data privacy regulations.

Throughout the workflow illustrated in FIG. 5, the platform can employ event-driven architectures and asynchronous message handling to ensure responsive interactions and reliable backend operations. Every user action—whether authentication, command execution, data retrieval, or process initiation—can be logged in a centralized audit trail. These logs can include timestamps, user identifiers, action types, data object references, and system responses, providing a comprehensive record of platform activity. Additionally, telemetry and analytics data can be collected to monitor usage patterns, detect anomalies, and improve future.

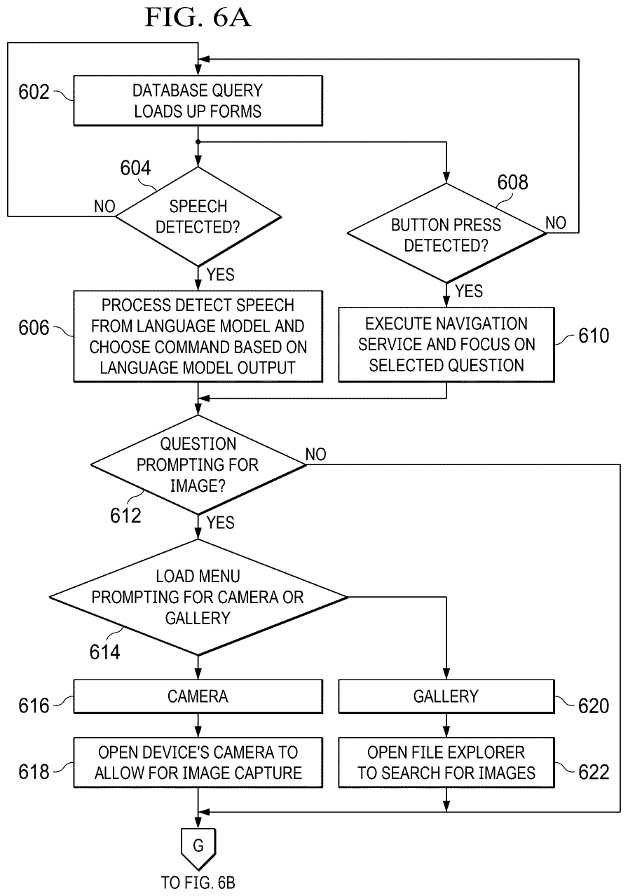

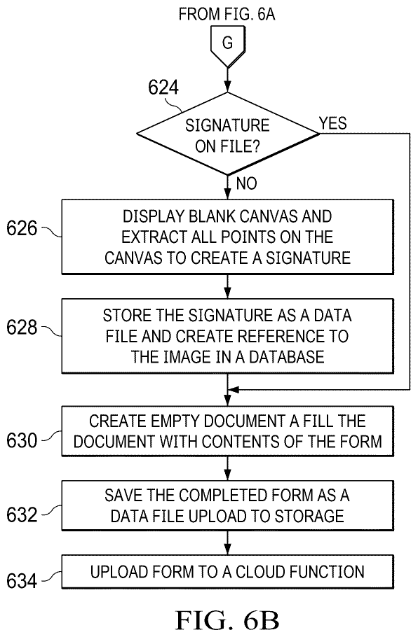

FIGS. 6A-6B illustrate an example flow diagram showing how the platform facilitates user interaction with digital forms, including uploading required information, capturing signatures, and generating finalized documents, all within an AI-assisted avatar workflow. The process integrates front-end user interactions with backend services such as database queries, NLP, image capture, and cloud storage operations to create a seamless and secure digital signing experience.

At stage 602, the platform can execute a database query to retrieve a form associated with a particular patient, procedure, or administrative task. This query can target a structured forms database, which stores templates for various document types (e.g., consent forms, medical history forms, HIPAA authorizations, financial agreements, etc.). Once retrieved, the form template and its metadata are loaded into the user interface and prepared for interactive completion. The backend system can also retrieve any previously submitted data relevant to the form, such as patient demographics or prior treatment details, to pre-populate fields where appropriate.

At stage 604, the platform can check for speech input from the user. If speech is detected, the audio is processed by a backend NLP pipeline. The audio stream is first converted to text using an ASR model, after which the textual data is passed to a language model through an API call. The language model interprets user intent and extracts relevant commands, such as navigation requests (“Next question”), content updates (“Change my address”), or submission confirmations (“I'm ready to sign”). Based on the processed input, the platform selects the appropriate action or command and executes it.

If no speech input is detected, the platform monitors for GUI-based interactions. At stage 608, if a button press is detected, then at stage 610 a navigation service can be triggered. This service can include logic for focusing on specific form sections, advancing to the next question, or dynamically expanding optional fields. Backend event handling services can record these interactions, updating session state and storing partial responses to ensure data persistence even if the session is interrupted.

In certain scenarios, the form may prompt the user to provide an image—for example, a scanned copy of an identification card or insurance documentation. At stage 612, the platform can check whether the current question requires image input. If so, the interface transitions to a media input mode. At stage 614, a menu can be displayed giving the user the option to capture a new image using the device's camera or upload an existing image from the device's gallery.

If camera is selected at stage 616, the platform can request access to the device's camera hardware and open a native camera interface at stage 618. The user can then capture a new image, which the platform automatically compresses, encodes, and uploads to a secure storage location. If gallery is selected at stage 620, the platform can launch the device's file explorer at stage 622, allowing the user to select an existing image. The backend validates the file format, performs optional preprocessing (e.g., orientation correction, metadata stripping, etc.), and links the image to the current form submission.

Following the media capture phase—or if no image is required—the platform can check at stage 624 whether a valid signature is already on file. This check can involve querying a signature database linked to the user's profile. If no signature exists, the platform can display a digital signature capture interface at stage 626. This interface can consist of a blank canvas where the user can draw their signature using a touchscreen, mouse, or stylus. The application captures a series of coordinate points representing the drawn signature, converts them into a scalable image object, and stores the resulting image in a secure signature repository. A reference pointer to this stored image is then created and associated with the user's record.

After a signature is confirmed—either retrieved from storage or newly captured—the platform can proceed to generate the final document. At stage 630, the platform can create a new document object and merge it with the contents of the form template. This process can include inserting user-supplied data, embedding uploaded images, and overlaying the digital signature in the appropriate location. The document composition engine can also apply formatting rules, pagination, headers, and metadata such as timestamps and audit trail identifiers.

Once the document is assembled, it is finalized and stored. At stage 632, the platform can save the completed document—which can be in a portable document format (PDF)—to a storage system such as Firebase Storage or an equivalent cloud-based repository. The platform can also store a structured representation of the user's responses in the platform's primary database, enabling future retrieval, indexing, and analytics.

Finally, at stage 634, the platform can pass the completed form to a backend cloud function responsible for further distribution. This function can upload the document to an easily accessible cloud folder, email it to authorized recipients, or integrate it with third-party EHR systems. The platform may also update the user's profile with metadata about the completed form, including the date, document type, and any associated workflows triggered by its completion (e.g., appointment scheduling, insurance verification, etc.).

In this way, FIGS. 6A-6B illustrate how the platform orchestrates a complete, end-to-end document execution process that leverages both natural language input and traditional user interface interactions. Through tightly integrated backend services—including NLP processing, database querying, image handling, digital signature capture, and cloud storage—the platform enables users to complete and sign complex forms in an intuitive, AI-assisted environment while maintaining strict data integrity, security, and compliance standards.

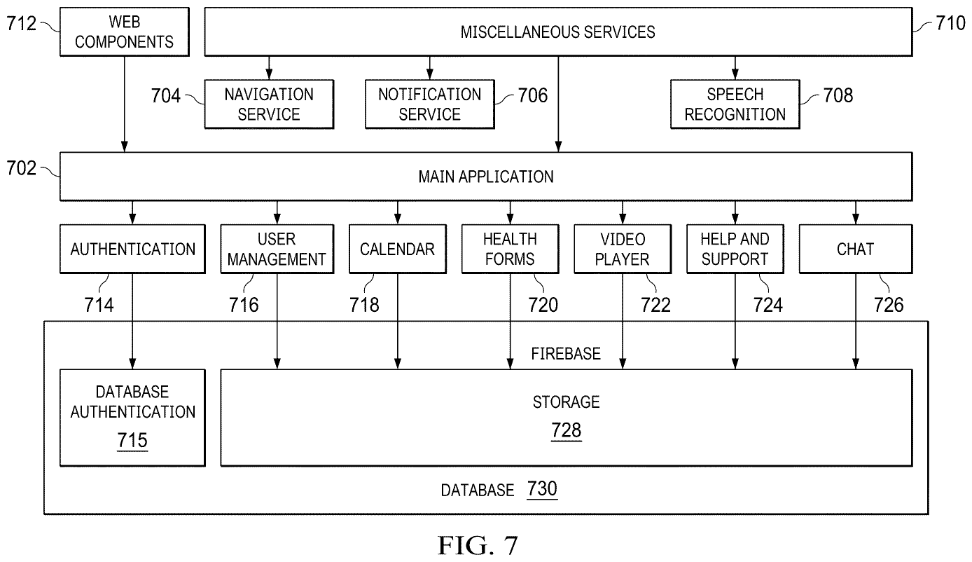

FIG. 7 illustrates a UML diagram that represents an example overall architecture and component relationships of the AI-assisted intake platform. The diagram shows how different software modules, services, and data layers interact to deliver the platform's functionality to end users, including patients, providers, and administrators. The structure is designed to modularize responsibilities, separate frontend and backend services, and enable scalable, event-driven communication among components.

At the center of the architecture is a main application 702, which functions as the core orchestration layer of the platform. The main application 702 coordinates communication between various frontend inputs, backend services, and data storage layers. It acts as the primary execution context for handling user requests, managing session state, routing service calls, and facilitating interactions between external services and internal subsystems. Incoming data and commands flow into the main application from multiple sources, including web components 712 and miscellaneous services 710. Web components 712 represent user interface elements rendered in the client application, such as form fields, navigation buttons, chat interfaces, or appointment widgets. These components capture user input and relay it to the main application 702 for processing. Miscellaneous services 710 encompass a range of utility and support services, including analytics engines, content delivery services, optical character recognition (OCR) tools, NLP modules, and external APIs that provide data enrichment or third-party integrations. The main application 702 also interfaces directly with three foundational system services: a navigation service 704, a notification service 706, and a speech recognition service 708. The navigation service 704 handles routing logic, view transitions, and context management within the user interface. The notification service 706 is responsible for delivering in-app alerts, push notifications, and scheduled reminders, while the speech recognition service 708 provides voice input processing capabilities, including audio-to-text conversion, command extraction, and intent detection.

The main application 702 also serves as the access point for a series of domain-specific modules that represent core features of the platform. These include authentication 714, user management 716, calendar 718, health forms 720, video player 722, help and support 724, and chat 726. Each module encapsulates a distinct functional area: the authentication 714 module manages user sign-in and identity verification workflows, while the user management 716 module stores and retrieves profile data and account settings. The calendar 718 module interfaces with scheduling services to retrieve, display, and modify appointment data, and the health forms 720 module handles the creation, population, and storage of digital intake forms. The video player 722 module enables playback of educational or consultative media content, the help and support 724 module provides access to documentation and user assistance tools, and the chat 726 module manages real-time messaging sessions between users and providers or administrative staff. Each of these modules communicates with backend infrastructure and external services through the main application 702, which standardizes message formats, enforces security policies, and maintains application state across user sessions.

The authentication 714 module has a direct dependency on database authentication 715, a dedicated service that handles credential validation, token generation, and secure session management. This service is part of a broader authentication framework provided by a backend application platform 730 (e.g., FIREBASE, etc.), which allows for multi-factor authentication, OAuth integration, and role-based access control. The remaining modules—user management 716, calendar 718, health forms 720, video player 722, help and support 724, and chat 726—are connected to storage 728, which serves as the primary data persistence layer. Storage 728, which can also be implemented using backend application platform 730, supports both structured and unstructured data storage, enabling the platform to manage diverse data types such as user profiles, form submissions, message logs, multimedia content, and system metadata. Data written to storage 728 can be indexed, queried, and retrieved by authorized services, with access governed by security rules and compliance constraints.

Overall, the platform architecture depicted in FIG. 7 demonstrates how the platform integrates user interfaces, backend services, and data layers into a cohesive application framework. The main application 702 functions as the central hub that orchestrates data flow between user-facing components, backend processing services, and persistent storage, ensuring that the platform remains modular, scalable, and capable of supporting complex workflows across patient, provider, and administrative contexts. This modular structure allows for independent development, deployment, and scaling of individual services while maintaining consistent communication patterns and data integrity throughout the system.

In the context of the present patent application, the term “connection,” the term “component” and/or similar terms are intended to be physical, but are not necessarily always tangible. Whether or not these terms refer to tangible subject matter, thus, may vary in a particular context of usage. As an example, a tangible connection and/or tangible connection path may be made, such as by a tangible, electrical connection, such as an electrically conductive path comprising metal or other conductor, that is able to conduct electrical current between two tangible components. Likewise, a tangible connection path may be at least partially affected and/or controlled, such that, as is typical, a tangible connection path may be open or closed, at times resulting from influence of one or more externally derived signals, such as external currents and/or voltages, such as for an electrical switch. Non-limiting illustrations of an electrical switch include a transistor, a diode, etc. However, a “connection” and/or “component,” in a particular context of usage, likewise, although physical, can also be non-tangible, such as a connection between a client and a server over a network, particularly a wireless network, which generally refers to the ability for the client and server to transmit, receive, and/or exchange communications, as discussed in more detail later.

In a particular context of usage, such as a particular context in which tangible components are being discussed, therefore, the terms “coupled” and “connected” are used in a manner so that the terms are not synonymous. Similar terms may also be used in a manner in which a similar intention is exhibited. Thus, “connected” is used to indicate that two or more tangible components and/or the like, for example, are tangibly in direct physical contact. Thus, using the previous example, two tangible components that are electrically connected are physically connected via a tangible electrical connection, as previously discussed. However, “coupled,” is used to mean that potentially two or more tangible components are tangibly in direct physical contact. Nonetheless, “coupled” is also used to mean that two or more tangible components and/or the like are not necessarily tangibly in direct physical contact, but are able to co-operate, liaise, and/or interact, such as, for example, by being “optically coupled.” Likewise, the term “coupled” is also understood to mean indirectly connected. It is further noted, in the context of the present patent application, since memory, such as a memory component and/or memory states, is intended to be non-transitory, the term physical, at least if used in relation to memory necessarily implies that such memory components and/or memory states, continuing with the example, are tangible.

Additionally, in the present patent application, in a particular context of usage, such as a situation in which tangible components (and/or similarly, tangible materials) are being discussed, a distinction exists between being “on” and being “over.” As an example, deposition of a substance “on” a substrate refers to a deposition involving direct physical and tangible contact without an intermediary, such as an intermediary substance, between the substance deposited and the substrate in this latter example; nonetheless, deposition “over” a substrate, while understood to potentially include deposition “on” a substrate (since being “on” may also accurately be described as being “over”), is understood to include a situation in which one or more intermediaries, such as one or more intermediary substances, are present between the substance deposited and the substrate so that the substance deposited is not necessarily in direct physical and tangible contact with the substrate.

A similar distinction is made in an appropriate particular context of usage, such as in which tangible materials and/or tangible components are discussed, between being “beneath” and being “under.” While “beneath,” in such a particular context of usage, is intended to necessarily imply physical and tangible contact (similar to “on,” as just described), “under” potentially includes a situation in which there is direct physical and tangible contact, but does not necessarily imply direct physical and tangible contact, such as if one or more intermediaries, such as one or more intermediary substances, are present. Thus, “on” is understood to mean “immediately over” and “beneath” is understood to mean “immediately under.”

It is likewise appreciated that terms such as “over” and “under” are understood in a similar manner as the terms “up,” “down,” “top,” “bottom,” and so on, previously mentioned. These terms may be used to facilitate discussion, but are not intended to necessarily restrict scope of claimed subject matter. For example, the term “over,” as an example, is not meant to suggest that claim scope is limited to only situations in which an embodiment is right side up, such as in comparison with the embodiment being upside down, for example. An example includes a flip chip, as one illustration, in which, for example, orientation at various times (e.g., during fabrication, etc.) may not necessarily correspond to orientation of a final product. Thus, if an object, as an example, is within applicable claim scope in a particular orientation, such as upside down, as one example, likewise, it is intended that the latter also be interpreted to be included within applicable claim scope in another orientation, such as right side up, again, as an example, and vice-versa, even if applicable literal claim language has the potential to be interpreted otherwise. Of course, again, as always has been the case in the specification of a patent application, particular context of description and/or usage provides helpful guidance regarding reasonable inferences to be drawn.

Unless otherwise indicated, in the context of the present patent application, the term “or” if used to associate a list, such as A, B, or C, is intended to mean A, B, and C, here used in the inclusive sense, as well as A, B, or C, here used in the exclusive sense. With this understanding, “and” is used in the inclusive sense and intended to mean A, B, and C; whereas “and/or” can be used in an abundance of caution to make clear that all of the foregoing meanings are intended, although such usage is not required. In addition, the term “one or more” and/or similar terms is used to describe any feature, structure, characteristic, and/or the like in the singular, “and/or” is also used to describe a plurality and/or some other combination of features, structures, characteristics, and/or the like. Likewise, the term “based on” and/or similar terms are understood as not necessarily intending to convey an exhaustive list of factors, but to allow for existence of additional factors not necessarily expressly described.

Furthermore, it is intended, for a situation that relates to implementation of claimed subject matter and is subject to testing, measurement, and/or specification regarding degree, that the particular situation be understood in the following manner. As an example, in a given situation, assume a value of a physical property is to be measured. If alternatively reasonable approaches to testing, measurement, and/or specification regarding degree, at least with respect to the property, continuing with the example, is reasonably likely to occur to one of ordinary skill, at least for implementation purposes, claimed subject matter is intended to cover those alternatively reasonable approaches unless otherwise expressly indicated. As an example, if a plot of measurements over a region is produced and implementation of claimed subject matter refers to employing a measurement of slope over the region, but a variety of reasonable and alternative techniques to estimate the slope over that region exist, claimed subject matter is intended to cover those reasonable alternative techniques unless otherwise expressly indicated.

To the extent claimed subject matter is related to one or more particular measurements, such as with regard to physical manifestations capable of being measured physically, such as, without limit, temperature, pressure, voltage, current, electromagnetic radiation, etc., it is believed that claimed subject matter does not fall within the abstract idea judicial exception to statutory subject matter. Rather, it is asserted, that physical measurements are not mental steps and, likewise, are not abstract ideas.

It is noted, nonetheless, that a typical measurement model employed is that one or more measurements may respectively comprise a sum of at least two components. Thus, for a given measurement, for example, one component may comprise a deterministic component, which in an ideal sense, may comprise a physical value (e.g., sought via one or more measurements, etc.), often in the form of one or more signals, signal samples and/or states, and one component may comprise a random component, which may have a variety of sources that may be challenging to quantify. At times, for example, lack of measurement precision may affect a given measurement. Thus, for claimed subject matter, a statistical or stochastic model may be used in addition to a deterministic model as an approach to identification and/or prediction regarding one or more measurement values that may relate to claimed subject matter.

For example, a relatively large number of measurements may be collected to better estimate a deterministic component. Likewise, if measurements vary, which may typically occur, it may be that some portion of a variance may be explained as a deterministic component, while some portion of a variance may be explained as a random component. Typically, it is desirable to have stochastic variance associated with measurements be relatively small, if feasible. That is, typically, it may be preferable to be able to account for a reasonable portion of measurement variation in a deterministic manner, rather than a stochastic matter as an aid to identification and/or predictability.

Along these lines, a variety of techniques have come into use so that one or more measurements may be processed to better estimate an underlying deterministic component, as well as to estimate potentially random components. These techniques, of course, may vary with details surrounding a given situation. Typically, however, more complex problems may involve use of more complex techniques. In this regard, as alluded to above, one or more measurements of physical manifestations may be modelled deterministically and/or stochastically. Employing a model permits collected measurements to potentially be identified and/or processed, and/or potentially permits estimation and/or prediction of an underlying deterministic component, for example, with respect to later measurements to be taken. A given estimate may not be a perfect estimate; however, in general, it is expected that on average one or more estimates may better reflect an underlying deterministic component, for example, if random components that may be included in one or more obtained measurements, are considered. Practically speaking, of course, it is desirable to be able to generate, such as through estimation approaches, a physically meaningful model of processes affecting measurements to be taken.

In some situations, however, as indicated, potential influences may be complex. Therefore, seeking to understand appropriate factors to consider may be particularly challenging. In such situations, it is, therefore, not unusual to employ heuristics with respect to generating one or more estimates. Heuristics refers to use of experience related approaches that may reflect realized processes and/or realized results, such as with respect to use of historical measurements, for example. Heuristics, for example, may be employed in situations where more analytical approaches may be overly complex and/or nearly intractable. Thus, regarding claimed subject matter, an innovative feature may include, in an example embodiment, heuristics that may be employed, for example, to estimate and/or predict one or more measurements.

It is further noted that the terms “type” and/or “like,” if used, such as with a feature, structure, characteristic, and/or the like, using “optical” or “electrical” as simple examples, means at least partially of and/or relating to the feature, structure, characteristic, and/or the like in such a way that presence of minor variations, even variations that might otherwise not be considered fully consistent with the feature, structure, characteristic, and/or the like, do not in general prevent the feature, structure, characteristic, and/or the like from being of a “type” and/or being “like,” (such as being an “optical-type” or being “optical-like,” for example) if the minor variations are sufficiently minor so that the feature, structure, characteristic, and/or the like would still be considered to be substantially present with such variations also present. Thus, continuing with this example, the terms optical-type and/or optical-like properties are necessarily intended to include optical properties. Likewise, the terms electrical-type and/or electrical-like properties, as another example, are necessarily intended to include electrical properties. It should be noted that the specification of the present patent application merely provides one or more illustrative examples and claimed subject matter is intended to not be limited to one or more illustrative examples; however, again, as has always been the case with respect to the specification of a patent application, particular context of description and/or usage provides helpful guidance regarding reasonable inferences to be drawn.

With advances in technology, it has become more typical to employ distributed computing and/or communication approaches in which portions of a process, such as signal processing of signal samples, for example, may be allocated among various devices, including one or more client devices and/or one or more server devices, via a computing and/or communications network, for example. A network may comprise two or more devices, such as network devices and/or computing devices, and/or may couple devices, such as network devices and/or computing devices, so that signal communications, such as in the form of signal packets and/or signal frames (e.g., comprising one or more signal samples, etc.), for example, may be exchanged, such as between a server device and/or a client device, as well as other types of devices, including between wired and/or wireless devices coupled via a wired and/or wireless network, for example.

An example of a distributed computing system comprises the so-called Hadoop distributed computing system, which employs a map-reduce type of architecture. In the context of the present patent application, the terms map-reduce architecture and/or similar terms are intended to refer to a distributed computing system implementation and/or embodiment for processing and/or for generating larger sets of signal samples employing map and/or reduce operations for a parallel, distributed process performed over a network of devices. A map operation and/or similar terms refer to processing of signals (e.g., signal samples, etc.) to generate one or more key-value pairs and to distribute the one or more pairs to one or more devices of the system (e.g., network, etc.). A reduce operation and/or similar terms refer to processing of signals (e.g., signal samples, etc.) via a summary operation (e.g., such as counting the number of students in a queue, yielding name frequencies, etc.). A system may employ such an architecture, such as by marshaling distributed server devices, executing various tasks in parallel, and/or managing communications, such as signal transfers, between various parts of the system (e.g., network), etc., in an embodiment. As mentioned, one non-limiting, but well-known, example comprises the Hadoop distributed computing system. It refers to an open source implementation and/or embodiment of a map-reduce type architecture (, but may include other aspects, such as the Hadoop distributed file system (HDFS). In general, therefore, “Hadoop” and/or similar terms (e.g., “Hadoop-type,” etc.) refer to an implementation and/or embodiment of a scheduler for executing larger processing jobs using a map-reduce architecture over a distributed system. Furthermore, in the context of the present patent application, use of the term “Hadoop” is intended to include versions, presently known and/or to be later developed.

In the context of the present patent application, the term network device refers to any device capable of communicating via and/or as part of a network and may comprise a computing device. While network devices may be capable of communicating signals (e.g., signal packets and/or frames, etc.), such as via a wired and/or wireless network, they may also be capable of performing operations associated with a computing device, such as arithmetic and/or logic operations, processing and/or storing operations (e.g., storing signal samples, etc.), such as in memory as tangible, physical memory states, and/or may, for example, operate as a server device and/or a client device in various embodiments. Network devices capable of operating as a server device, a client device and/or otherwise, may include, as examples, dedicated rack-mounted servers, desktop computers, laptop computers, set top boxes, tablets, netbooks, smart phones, wearable devices, integrated devices combining two or more features of the foregoing devices, and/or the like, or any combination thereof. As mentioned, signal packets and/or frames, for example, may be exchanged, such as between a server device and/or a client device, as well as other types of devices, including between wired and/or wireless devices coupled via a wired and/or wireless network, for example, or any combination thereof. It is noted that the terms, server, server device, server computing device, server computing platform and/or similar terms are used interchangeably. Similarly, the terms client, client device, client computing device, client computing platform and/or similar terms are also used interchangeably. While in some instances, for ease of description, these terms may be used in the singular, such as by referring to a “client device” or a “server device,” the description is intended to encompass one or more client devices and/or one or more server devices, as appropriate. Along similar lines, references to a “database” are understood to mean, one or more databases and/or portions thereof, as appropriate.

It should be understood that for ease of description, a network device (also referred to as a networking device) may be embodied and/or described in terms of a computing device and vice-versa. However, it should further be understood that this description should in no way be construed so that claimed subject matter is limited to one embodiment, such as only a computing device and/or only a network device, but, instead, may be embodied as a variety of devices or combinations thereof, including, for example, one or more illustrative examples.

A network may also include now known, and/or to be later developed arrangements, derivatives, and/or improvements, including, for example, past, present and/or future mass storage, such as network attached storage (NAS), a storage area network (SAN), and/or other forms of device readable media, for example. A network may include a portion of the Internet, one or more local area networks (LANs), one or more wide area networks (WANs), wire-line type connections, wireless type connections, other connections, or any combination thereof. Thus, a network may be worldwide in scope and/or extent. Likewise, sub-networks, such as may employ differing architectures and/or may be substantially compliant and/or substantially compatible with differing protocols, such as network computing and/or communications protocols (e.g., network protocols, etc.), may interoperate within a larger network.

In the context of the present patent application, the term sub-network and/or similar terms, if used, for example, with respect to a network, refers to the network and/or a part thereof. Sub-networks may also comprise links, such as physical links, connecting and/or coupling nodes, so as to be capable to communicate signal packets and/or frames between devices of particular nodes, including via wired links, wireless links, or combinations thereof. Various types of devices, such as network devices and/or computing devices, may be made available so that device interoperability is enabled and/or, in at least some instances, may be transparent. In the context of the present patent application, the term “transparent,” if used with respect to devices of a network, refers to devices communicating via the network in which the devices are able to communicate via one or more intermediate devices, such as one or more intermediate nodes, but without the communicating devices necessarily specifying the one or more intermediate nodes and/or the one or more intermediate devices of the one or more intermediate nodes and/or, thus, may include within the network the devices communicating via the one or more intermediate nodes and/or the one or more intermediate devices of the one or more intermediate nodes, but may engage in signal communications as if such intermediate nodes and/or intermediate devices are not necessarily involved. For example, a router may provide a link and/or connection between otherwise separate and/or independent LANs.

In the context of the present patent application, a “private network” refers to a particular, limited set of devices, such as network devices and/or computing devices, able to communicate with other devices, such as network devices and/or computing devices, in the particular, limited set, such as via signal packet and/or signal frame communications, for example, without a need for re-routing and/or redirecting signal communications. A private network may comprise a stand-alone network; however, a private network may also comprise a subset of a larger network, such as, for example, without limitation, all or a portion of the Internet. Thus, for example, a private network “in the cloud” may refer to a private network that comprises a subset of the Internet. Although signal packet and/or frame communications (e.g. signal communications, etc.) may employ intermediate devices of intermediate nodes to exchange signal packets and/or signal frames, those intermediate devices may not necessarily be included in the private network by not being a source or designated destination for one or more signal packets and/or signal frames, for example. It is understood in the context of the present patent application that a private network may direct outgoing signal communications to devices not in the private network, but devices outside the private network may not necessarily be able to direct inbound signal communications to devices included in the private network.

The Internet refers to a decentralized global network of interoperable networks that comply with the IP. It is noted that there are several versions of the Internet Protocol. The term Internet Protocol, IP, and/or similar terms are intended to refer to any version, now known and/or to be later developed. The Internet includes LANs, WANs, wireless networks, and/or long haul public networks that, for example, may allow signal packets and/or frames to be communicated between LANs. The term World Wide Web (WWW or Web) and/or similar terms may also be used, although it refers to a part of the Internet that complies with the Hypertext Transfer Protocol (HTTP). For example, network devices may engage in an HTTP session through an exchange of appropriately substantially compatible and/or substantially compliant signal packets and/or frames. It is noted that there are several versions of the Hypertext Transfer Protocol. The term Hypertext Transfer Protocol, HTTP, and/or similar terms are intended to refer to any version, now known and/or to be later developed. It is likewise noted that in various places in this document substitution of the term Internet with the term World Wide Web (“Web”) may be made without a significant departure in meaning and may, therefore, also be understood in that manner if the statement would remain correct with such a substitution.

Although claimed subject matter is not in particular limited in scope to the Internet and/or to the Web; nonetheless, the Internet and/or the Web may without limitation provide a useful example of an embodiment at least for purposes of illustration. As indicated, the Internet and/or the Web may comprise a worldwide system of interoperable networks, including interoperable devices within those networks. The Internet and/or Web has evolved to a public, self-sustaining facility accessible to potentially billions of people or more worldwide. Also, in an embodiment, and as mentioned above, the terms “WWW” and/or “Web” refer to a part of the Internet that complies with the Hypertext Transfer Protocol. The Internet and/or the Web, therefore, in the context of the present patent application, may comprise a service that organizes stored digital content, such as, for example, text, images, video, etc., through the use of hypermedia, for example. It is noted that a network, such as the Internet and/or Web, may be employed to store electronic files and/or electronic documents.

The term electronic file and/or the term electronic document are used throughout this document to refer to a set of stored memory states and/or a set of physical signals associated in a manner so as to thereby at least logically form a file (e.g., electronic, etc.) and/or an electronic document. That is, it is not meant to implicitly reference a particular syntax, format and/or approach used, for example, with respect to a set of associated memory states and/or a set of associated physical signals. If a particular type of file storage format and/or syntax, for example, is intended, it is referenced expressly. It is further noted an association of memory states, for example, may be in a logical sense and not necessarily in a tangible, physical sense. Thus, although signal and/or state components of a file and/or an electronic document, for example, are to be associated logically, storage thereof, for example, may reside in one or more different places in a tangible, physical memory, in an embodiment.

A Hyper Text Markup Language (“HTML”), for example, may be utilized, in whole or in part, to specify digital content and/or to specify a format thereof, such as in the form of an electronic file and/or an electronic document, such as a Web page, Web site, etc., for example. An Extensible Markup Language (“XML”) may also be utilized, in whole or in part, to specify digital content and/or to specify a format thereof, such as in the form of an electronic file and/or an electronic document, such as a Web page, Web site, etc., in an embodiment. Of course, HTML and/or XML are merely examples of “markup” languages, provided as non-limiting illustrations. Furthermore, HTML and/or XML are intended to refer to any version, now known and/or to be later developed, of these languages. Likewise, claimed subject matter are not intended to be limited to examples provided as illustrations, of course.

In the context of the present patent application, the term “Web site” and/or similar terms refer to Web pages that are associated electronically to form a particular collection thereof. Also, in the context of the present patent application, “Web page” and/or similar terms refer to an electronic file and/or an electronic document accessible via a network, including by specifying a uniform resource locator (URL) for accessibility via the Web, in an example embodiment. As alluded to above, in one or more embodiments, a Web page may comprise digital content coded (e.g., via computer instructions, etc.) using one or more languages, such as, for example, markup languages, including HTML and/or XML, although claimed subject matter is not limited in scope in this respect. Also, in one or more embodiments, application developers may write code (e.g., computer instructions, etc.) in the form of JavaScript (or other programming languages), for example, executable by a computing device to provide digital content to populate an electronic document and/or an electronic file in an appropriate format, such as for use in a particular application, for example. Use of the term “JavaScript” and/or similar terms intended to refer to one or more particular programming languages are intended to refer to any version of the one or more programming languages identified, now known and/or to be later developed. Thus, JavaScript is merely an example programming language. As was mentioned, claimed subject matter is not intended to be limited to examples and/or illustrations.

In the context of the present patent application, the terms “entry,” “electronic entry,” “document,” “electronic document,” “content,”, “digital content,” “item,” and/or similar terms are meant to refer to signals and/or states in a physical format, such as a digital signal and/or digital state format, e.g., that may be perceived by a user if displayed, played, tactilely generated, etc. and/or otherwise executed by a device, such as a digital device, including, for example, a computing device, but otherwise might not necessarily be readily perceivable by humans (e.g., if in a digital format, etc.). Likewise, in the context of the present patent application, digital content provided to a user in a form so that the user is able to readily perceive the underlying content itself (e.g., content presented in a form consumable by a human, such as hearing audio, feeling tactile sensations and/or seeing images, as examples) is referred to, with respect to the user, as “consuming” digital content, “consumption” of digital content, “consumable” digital content and/or similar terms. For one or more embodiments, an electronic document and/or an electronic file may comprise a Web page of code (e.g., computer instructions, etc.) in a markup language executed or to be executed by a computing and/or networking device, for example. In another embodiment, an electronic document and/or electronic file may comprise a portion and/or a region of a Web page. However, claimed subject matter is not intended to be limited in these respects.

Also, for one or more embodiments, an electronic document and/or electronic file may comprise a number of components. As previously indicated, in the context of the present patent application, a component is physical, but is not necessarily tangible. As an example, components with reference to an electronic document and/or electronic file, in one or more embodiments, may comprise text, for example, in the form of physical signals and/or physical states (e.g., capable of being physically displayed, etc.). Typically, memory states, for example, comprise tangible components, whereas physical signals are not necessarily tangible, although signals may become (e.g., be made, etc.) tangible, such as if appearing on a tangible display, for example, as is not uncommon. Also, for one or more embodiments, components with reference to an electronic document and/or electronic file may comprise a graphical object, such as, for example, an image, such as a digital image, and/or sub-objects, including attributes thereof, which, again, comprise physical signals and/or physical states (e.g., capable of being tangibly displayed, etc.). In an embodiment, digital content may comprise, for example, text, images, audio, video, and/or other types of electronic documents and/or electronic files, including portions thereof, for example.

Also, in the context of the present patent application, the term parameters (e.g., one or more parameters, etc.) refer to material descriptive of a collection of signal samples, such as one or more electronic documents and/or electronic files, and exist in the form of physical signals and/or physical states, such as memory states. For example, one or more parameters, such as referring to an electronic document and/or an electronic file comprising an image, may include, as examples, time of day at which an image was captured, latitude and longitude of an image capture device, such as a camera, for example, etc. In another example, one or more parameters relevant to digital content, such as digital content comprising a technical article, as an example, may include one or more authors, for example. Claimed subject matter is intended to embrace meaningful, descriptive parameters in any format, so long as the one or more parameters comprise physical signals and/or states, which may include, as parameter examples, collection name (e.g., electronic file and/or electronic document identifier name, etc.), technique of creation, purpose of creation, time and date of creation, logical path if stored, coding formats (e.g., type of computer instructions, such as a markup language, etc.) and/or standards and/or specifications used so as to be protocol compliant (e.g., meaning substantially compliant and/or substantially compatible, etc.) for one or more uses, and so forth.

Signal packet communications and/or signal frame communications, also referred to as signal packet transmissions and/or signal frame transmissions (or merely “signal packets” or “signal frames”), may be communicated between nodes of a network, where a node may comprise one or more network devices and/or one or more computing devices, for example. As an illustrative example, but without limitation, a node may comprise one or more sites employing a local network address, such as in a local network address space. Likewise, a device, such as a network device and/or a computing device, may be associated with that node. It is also noted that in the context of this patent application, the term “transmission” is intended as another term for a type of signal communication that may occur in any one of a variety of situations. Thus, it is not intended to imply a particular directionality of communication and/or a particular initiating end of a communication path for the “transmission” communication. For example, the mere use of the term in and of itself is not intended, in the context of the present patent application, to have particular implications with respect to the one or more signals being communicated, such as, for example, whether the signals are being communicated “to” a particular device, whether the signals are being communicated “from” a particular device, and/or regarding which end of a communication path may be initiating communication, such as, for example, in a “push type” of signal transfer or in a “pull type” of signal transfer. In the context of the present patent application, push and/or pull type signal transfers are distinguished by which end of a communications path initiates signal transfer.

Thus, a signal packet and/or frame may, as an example, be communicated via a communication channel and/or a communication path, such as comprising a portion of the Internet and/or the Web, from a site via an access node coupled to the Internet or vice-versa. Likewise, a signal packet and/or frame may be forwarded via network nodes to a target site coupled to a local network, for example. A signal packet and/or frame communicated via the Internet and/or the Web, for example, may be routed via a path, such as either being “pushed” or “pulled,” comprising one or more gateways, servers, etc. that may, for example, route a signal packet and/or frame, such as, for example, substantially in accordance with a target and/or destination address and availability of a network path of network nodes to the target and/or destination address. Although the Internet and/or the Web comprise a network of interoperable networks, not all of those interoperable networks are necessarily available and/or accessible to the public.

In the context of the particular patent application, a network protocol, such as for communicating between devices of a network, may be characterized, at least in part, substantially in accordance with a layered description, such as the so-called Open Systems Interconnection (OSI) seven layer type of approach and/or description. A network computing and/or communications protocol (also referred to as a network protocol) refers to a set of signaling conventions, such as for communication transmissions, for example, as may take place between and/or among devices in a network. In the context of the present patent application, the term “between” and/or similar terms are understood to include “among” if appropriate for the particular usage and vice-versa. Likewise, in the context of the present patent application, the terms “compatible with,” “comply with” and/or similar terms are understood to respectively include substantial compatibility and/or substantial compliance.

A network protocol, such as protocols characterized substantially in accordance with the aforementioned OSI description, has several layers. These layers are referred to as a network stack. Various types of communications (e.g., transmissions, etc.), such as network communications, may occur across various layers. A lowest level layer in a network stack, such as the so-called physical layer, may characterize how symbols (e.g., bits and/or bytes, etc.) are communicated as one or more signals (and/or signal samples) via a physical medium (e.g., twisted pair copper wire, coaxial cable, fiber optic cable, wireless air interface, combinations thereof, etc.). Progressing to higher-level layers in a network protocol stack, additional operations and/or features may be available via engaging in communications that are substantially compatible and/or substantially compliant with a particular network protocol at these higher-level layers. For example, higher-level layers of a network protocol may, for example, affect device permissions, user permissions, etc.

A network and/or sub-network, in an embodiment, may communicate via signal packets and/or signal frames, such as via participating digital devices and may be substantially compliant and/or substantially compatible with, but is not limited to, now known and/or to be developed, versions of any of the following network protocol stacks: ARCNET, AppleTalk, ATM, Bluetooth, DECnet, Ethernet, FDDI, Frame Relay, HIPPI, IEEE 1394, IEEE 802.11, IEEE-488, Internet Protocol Suite, IPX, Myrinet, OSI Protocol Suite, QsNet, RS-232, SPX, System Network Architecture, Token Ring, USB, and/or X.25. A network and/or sub-network may employ, for example, a version, now known and/or later to be developed, of the following: TCP/IP, UDP, DECnet, NetBEUI, IPX, AppleTalk and/or the like. Versions of the IP may include IPv4, IPv6, and/or other later to be developed versions.

Regarding aspects related to a network, including a communications and/or computing network, a wireless network may couple devices, including client devices, with the network. A wireless network may employ stand-alone, ad-hoc networks, mesh networks, Wireless LAN (WLAN) networks, cellular networks, and/or the like. A wireless network may further include a system of terminals, gateways, routers, and/or the like coupled by wireless radio links, and/or the like, which may move freely, randomly and/or organize themselves arbitrarily, such that network topology may change, at times even rapidly. A wireless network may further employ a plurality of network access technologies, including a version of Long Term Evolution (LTE), WLAN, Wireless Router (WR) mesh, 2nd, 3rd, or 4th generation (2G, 3G, 4G, or 5G) cellular technology and/or the like, whether currently known and/or to be later developed. Network access technologies may enable wide area coverage for devices, such as computing devices and/or network devices, with varying degrees of mobility, for example.

A network may enable radio frequency and/or other wireless type communications via a wireless network access technology and/or air interface, such as Global System for Mobile communication (GSM), Universal Mobile Telecommunications System (UMTS), General Packet Radio Services (GPRS), Enhanced Data GSM Environment (EDGE), 3GPP LTE, LTE Advanced, WCDMA, BLUETOOTH, UWB, 802.11b/g/n, and/or the like. A wireless network may include virtually any type of now known and/or to be developed wireless communication mechanism and/or wireless communications protocol by which signals may be communicated between devices, between networks, within a network, and/or the like, including the foregoing, of course.

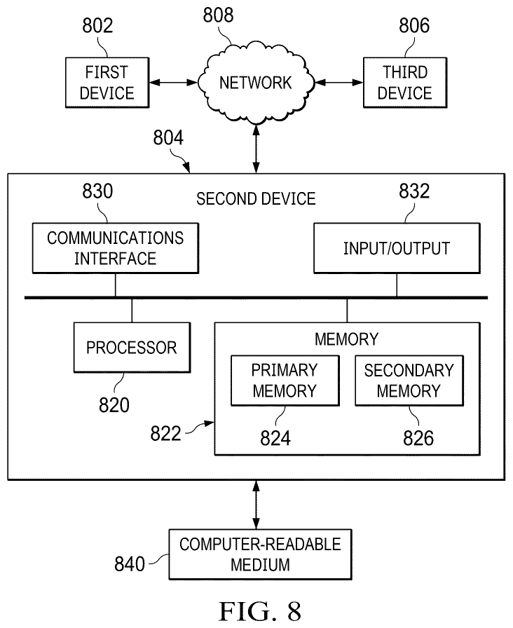

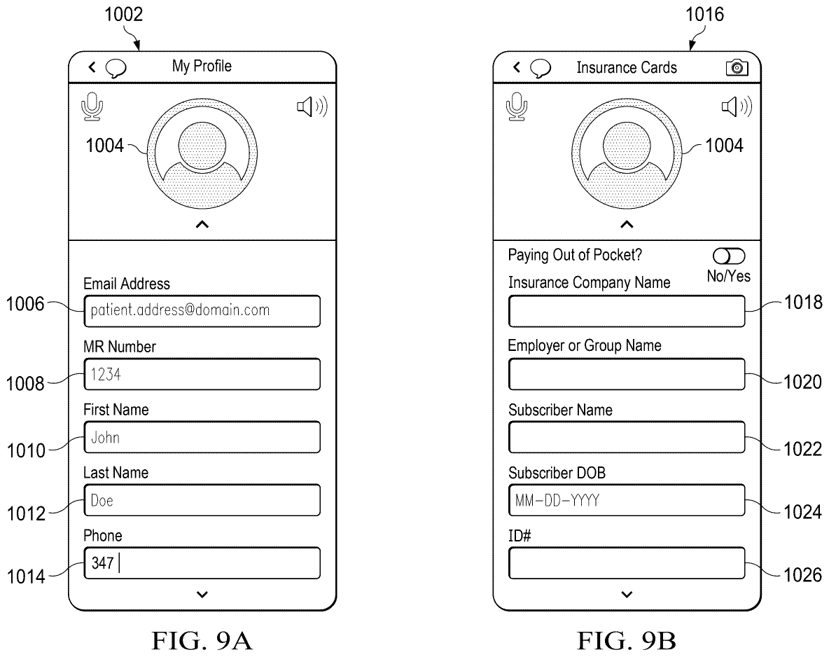

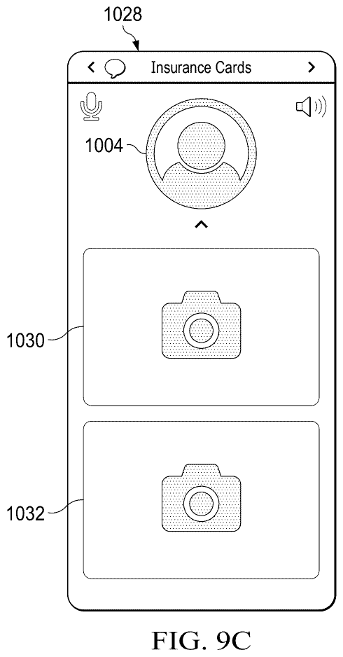

In one example embodiment, as shown in FIG. 8, a system embodiment may comprise a local network (e.g., device 804 and medium 840) and/or another type of network, such as a computing and/or communications network. For purposes of illustration, therefore, FIG. 8 shows an embodiment 800 of a system that may be employed to implement either type or both types of networks. Network 808 may comprise one or more network connections, links, processes, services, applications, and/or resources to facilitate and/or support communications, such as an exchange of communication signals, for example, between a computing device, such as 802, and another computing device, such as 806, which may, for example, comprise one or more client computing devices and/or one or more server computing device. By way of example, but not limitation, network 808 may comprise wireless and/or wired communication links, telephone and/or telecommunications systems, Wi-Fi networks, Wi-MAX networks, the Internet, a LAN, a WAN, or any combinations thereof.

Example devices in FIG. 8 may comprise features, for example, of a client computing device and/or a server computing device, in an embodiment. It is further noted that the term computing device, in general, whether employed as a client and/or as a server, or otherwise, refers at least to a processor and a memory connected by a communication bus. Likewise, in the context of the present patent application at least, this is understood to refer to sufficient structure within the meaning of 35 USC § 112 (f) so that it is specifically intended that 35 USC § 112 (f) not be implicated by use of the term “computing device” and/or similar terms; however, if it is determined, for some reason not immediately apparent, that the foregoing understanding cannot stand and that 35 USC § 112 (f), therefore, necessarily is implicated by the use of the term “computing device” and/or similar terms, then, it is intended, pursuant to that statutory section, that corresponding structure, material and/or acts for performing one or more functions be understood and be interpreted to be described at least in FIGS. 4-8 and/or FIGS. 10A-10C discussed herein, and in the text associated with the foregoing figure(s) of the present patent application.

Referring now to FIG. 8, in an embodiment, first and third devices 802 and 806 may be capable of rendering a GUI for a network device and/or a computing device, for example, so that a user-operator may engage in system use. Device 804 may potentially serve a similar function in this illustration. Likewise, in FIG. 8, computing device 802 (‘first device’ in figure) may interface with computing device 804 (‘second device’ in figure), which may, for example, also comprise features of a client computing device and/or a server computing device, in an embodiment. Processor (e.g., processing device, etc.) 820 and memory 822, which may comprise primary memory 824 and secondary memory 826, may communicate by way of a communication bus 815, for example. The term “computing device,” in the context of the present patent application, refers to a system and/or a device, such as a computing apparatus, that includes a capability to process (e.g., perform computations, etc.) and/or store digital content, such as electronic files, electronic documents, measurements, text, images, video, audio, etc. in the form of signals and/or states. Thus, a computing device, in the context of the present patent application, may comprise hardware, software, firmware, or any combination thereof (other than software per se). Computing device 804, as depicted in FIG. 8, is merely one example, and claimed subject matter is not limited in scope to this particular example.

For one or more embodiments, a device, such as a computing device and/or networking device, may comprise, for example, any of a wide range of digital electronic devices, including, but not limited to, desktop and/or notebook computers, high-definition televisions, digital versatile disc (DVD) and/or other optical disc players and/or recorders, game consoles, satellite television receivers, cellular telephones, tablet devices, wearable devices, personal digital assistants, mobile audio and/or video playback and/or recording devices, IoT type devices, or any combination of the foregoing. Further, unless specifically stated otherwise, a process as described, such as with reference to flow diagrams and/or otherwise, may also be executed and/or affected, in whole or in part, by a computing device and/or a network device. A device, such as a computing device and/or network device, may vary in terms of capabilities and/or features. Claimed subject matter is intended to cover a wide range of potential variations. For example, a device may include a numeric keypad and/or other display of limited functionality, such as a monochrome liquid crystal display (LCD) for displaying text, for example. In contrast, however, as another example, a web-enabled device may include a physical and/or a virtual keyboard, mass storage, one or more accelerometers, one or more gyroscopes, GPS and/or other location-identifying type capability, and/or a display with a higher degree of functionality, such as a touch-sensitive color 2D or 3D display, for example.

As suggested previously, communications between a computing device and/or a network device and a wireless network may be in accordance with known and/or to be developed network protocols including, for example, GSM, EDGE, 802.11b/g/n/h, etc., and/or worldwide interoperability for microwave access (WiMAX). A computing device and/or a networking device may also have a subscriber identity module (SIM) card, which, for example, may comprise a detachable or embedded smart card that is able to store subscription content of a user, and/or is also able to store a contact list. It is noted, however, that a SIM card may also be electronic, meaning that is may simply be stored in a particular location in memory of the computing and/or networking device. A user may own the computing device and/or network device or may otherwise be a user, such as a primary user, for example. A device may be assigned an address by a wireless network operator, a wired network operator, and/or an Internet Service Provider (ISP). For example, an address may comprise a domestic or international telephone number, an IP address, and/or one or more other identifiers. In other embodiments, a computing and/or communications network may be embodied as a wired network, wireless network, or any combinations thereof.

A computing and/or network device may include and/or may execute a variety of now known and/or to be developed operating systems, derivatives and/or versions thereof, including computer operating systems, such as Windows, iOS, Linux, a mobile operating system, such as iOS, Android, Windows Mobile, and/or the like. A computing device and/or network device may include and/or may execute a variety of possible applications, such as a client software application enabling communication with other devices. For example, one or more messages (e.g., content, etc.) may be communicated, such as via one or more protocols, now known and/or later to be developed, suitable for communication of email, short message service (SMS), and/or multimedia message service (MMS), including via a network, such as a social network, formed at least in part by a portion of a computing and/or communications network, including, but not limited to, Facebook, LinkedIn, Twitter, and/or Flickr, to provide only a few examples. A computing and/or network device may also include executable computer instructions to process and/or communicate digital content, such as, for example, textual content, digital multimedia content, and/or the like. A computing and/or network device may also include executable computer instructions to perform a variety of possible tasks, such as browsing, searching, playing various forms of digital content, including locally stored and/or streamed video, and/or games such as, but not limited to, fantasy sports leagues. The foregoing is provided merely to illustrate that claimed subject matter is intended to include a wide range of possible features and/or capabilities.

In FIG. 8, computing device 802 may provide one or more sources of executable computer instructions in the form physical states and/or signals (e.g., stored in memory states, etc.), for example. Computing device 802 may communicate with computing device 804 by way of a network connection, such as via network 808, for example. As previously mentioned, a connection, while physical, may not necessarily be tangible. Although computing device 804 of FIG. 8 shows various tangible, physical components, claimed subject matter is not limited to a computing devices having only these tangible components as other implementations and/or embodiments may include alternative arrangements that may comprise additional tangible components or fewer tangible components, for example, that function differently while achieving similar results. Rather, examples are provided merely as illustrations. It is not intended that claimed subject matter be limited in scope to illustrative examples.

Memory 822 may comprise any non-transitory storage mechanism. Memory 822 may comprise, for example, primary memory 824 and secondary memory 826, additional memory circuits, mechanisms, or combinations thereof may be used. Memory 822 may comprise, for example, random access memory, read only memory, etc., such as in the form of one or more storage devices and/or systems, such as, for example, a disk drive including an optical disc drive, a tape drive, a solid-state memory drive, etc., just to name a few examples.

Memory 822 may be utilized, in whole or in part, to store a program of executable computer instructions. For example, processor 820 may fetch executable instructions from memory and proceed to execute the fetched instructions. Memory 822 may also comprise a memory controller for accessing device readable-medium 840 that may carry and/or make accessible digital content, which may include code, and/or instructions, for example, executable by processor 820 and/or some other device, such as a controller, as one example, capable of executing computer instructions, for example. Under direction of processor 820, a non-transitory memory, such as memory cells storing physical states (e.g., memory states, etc.), comprising, for example, a program of executable computer instructions, may be executed by processor 820 and able to generate signals to be communicated via a network, for example, as previously described. Generated signals may also be stored in memory, also previously suggested.

Memory 822 may store electronic files and/or electronic documents, such as relating to one or more users, and may also comprise a computer-readable medium that may carry and/or make accessible content, including code and/or instructions, for example, executable by processor 820 and/or some other device, such as a controller, as one example, capable of executing computer instructions, for example. As previously mentioned, the term electronic file and/or the term electronic document are used throughout this document to refer to a set of stored memory states and/or a set of physical signals associated in a manner so as to thereby form an electronic file and/or an electronic document. That is, it is not meant to implicitly reference a particular syntax, format and/or approach used, for example, with respect to a set of associated memory states and/or a set of associated physical signals. It is further noted an association of memory states, for example, may be in a logical sense and not necessarily in a tangible, physical sense. Thus, although signal and/or state components of an electronic file and/or electronic document, are to be associated logically, storage thereof, for example, may reside in one or more different places in a tangible, physical memory, in an embodiment.

Algorithmic descriptions and/or symbolic representations are examples of techniques used by those of ordinary skill in the signal processing and/or related arts to convey the substance of their work to others skilled in the art. An algorithm is, in the context of the present patent application, and generally, is considered to be a self-consistent sequence of operations and/or similar signal processing leading to a desired result. In the context of the present patent application, operations and/or processing involve physical manipulation of physical quantities. Typically, although not necessarily, such quantities may take the form of electrical and/or magnetic signals and/or states capable of being stored, transferred, combined, compared, processed and/or otherwise manipulated, for example, as electronic signals and/or states making up components of various forms of digital content, such as signal measurements, text, images, video, audio, etc.

It has proven convenient at times, principally for reasons of common usage, to refer to such physical signals and/or physical states as bits, values, elements, parameters, symbols, characters, terms, numbers, numerals, measurements, content and/or the like. It should be understood, however, that all of these and/or similar terms are to be associated with appropriate physical quantities and are merely convenient labels. Unless specifically stated otherwise, as apparent from the preceding discussion, it is appreciated that throughout this specification discussions utilizing terms such as “processing,” “computing,” “calculating,” “determining”, “establishing”, “obtaining”, “identifying”, “selecting”, “generating”, and/or the like may refer to actions and/or processes of a specific apparatus, such as a special purpose computer and/or a similar special purpose computing and/or network device. In the context of this specification, therefore, a special purpose computer and/or a similar special purpose computing and/or network device is capable of processing, manipulating and/or transforming signals and/or states, typically in the form of physical electronic and/or magnetic quantities, within memories, registers, and/or other storage devices, processing devices, and/or display devices of the special purpose computer and/or similar special purpose computing and/or network device. In the context of this particular patent application, as mentioned, the term “specific apparatus” therefore includes a general purpose computing and/or network device, such as a general purpose computer, once it is programmed to perform particular functions, such as pursuant to program software instructions.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and/or storage of charge or a release of stored charge. Likewise, in other memory devices, a change of state may comprise a physical change, such as a transformation in magnetic orientation. Likewise, a physical change may comprise a transformation in molecular structure, such as from crystalline form to amorphous form or vice-versa. In still other memory devices, a change in physical state may involve quantum mechanical phenomena, such as, superposition, entanglement, and/or the like, which may involve quantum bits (qubits), for example. The foregoing is not intended to be an exhaustive list of all examples in which a change in state from a binary one to a binary zero or vice-versa in a memory device may comprise a transformation, such as a physical, but non-transitory, transformation. Rather, the foregoing is intended as illustrative examples.

Referring again to FIG. 8, processor 820 may comprise one or more circuits, such as digital circuits, to perform at least a portion of a computing procedure and/or process. By way of example, but not limitation, processor 820 may comprise one or more processors, such as controllers, microprocessors, microcontrollers, application specific integrated circuits, digital signal processors, programmable logic devices, field programmable gate arrays, the like, or any combination thereof. In various implementations and/or embodiments, processor 820 may perform signal processing, typically substantially in accordance with fetched executable computer instructions, such as to manipulate signals and/or states, to construct signals and/or states, etc., with signals and/or states generated in such a manner to be communicated and/or stored in memory, for example.

FIG. 8 also illustrates device 804 as including a component 832 operable with input/output devices, for example, so that signals and/or states may be appropriately communicated between devices, such as device 804 and an input device and/or device 804 and an output device. A user may make use of an input device, such as a computer mouse, stylus, track ball, keyboard, and/or any other similar device capable of receiving user actions and/or motions as input signals. Likewise, for a device having speech to text capability, a user may speak to a device to generate input signals. A user may make use of an output device, such as a display, a printer, etc., and/or any other device capable of providing signals and/or generating stimuli for a user, such as visual stimuli, audio stimuli and/or other similar stimuli.

FIGS. 10A-10D are illustrations depicting an example GUI for providing a virtual assistant during a digital intake process. FIG. 10A illustrates a personal information window 1002, which serves as an initial data collection page for gathering basic identifying information from a user, such as a patient accessing a dental or oral surgery intake platform. The personal information window 1002 includes a virtual avatar 1004, which functions as an interactive AI-assisted guide throughout the intake process. The avatar 1004 can be presented as an animated two-or three-dimensional character and may include synchronized voice output, lip movement, and facial expressions that correspond to spoken prompts generated by a text-to-speech model.

The avatar 1004 can assist the user by verbally prompting for required information, providing context for each data field, and confirming entries once they are submitted. For example, the avatar can audibly request that the user enter an email address into an email address field 1006, provide a medical record (MR) number 1008, and supply a first name 1010, last name 1012, and phone number 1014. In some embodiments, the avatar can accept natural language input via speech recognition and interpret commands using a NLU model, allowing the user to dictate responses rather than typing them. As data is entered, the avatar can provide feedback, such as indicating if a field has been successfully completed, suggesting corrections for improperly formatted inputs (e.g., a missing “@” symbol in an email address, etc.), or prompting the user to continue to the next step.

On the backend, the GUI is supported by several coordinated services that manage data flow and user interaction. If the page loads, a form management service can query a database to retrieve field definitions, validation rules, and user-specific metadata to pre-fill known values. As the user provides responses, a frontend event handler can validate each entry locally before transmitting the data to a backend API, where it is securely stored in an intake database associated with the patient's account. The avatar's dialogue flow can be dynamically generated by a dialogue management engine, which uses session state information to determine which prompt or instruction to deliver next. In some implementations, the backend may also track user progress and completion times to optimize future intake sessions or trigger automated workflows, such as verifying an email address or associating the MR number with existing patient records.

FIG. 10B illustrates an example insurance information window 1016 presented as part of the AI-assisted intake workflow. This interface is configured to collect insurance-related data from the user and may be displayed following completion of the personal information window described in connection with FIG. 10A. As shown, the insurance information window 1016 can include the same AI-assisted avatar 1004, which continues to guide the user through the data entry process by providing verbal prompts, contextual explanations, and confirmation feedback. The avatar 1004 may, for example, ask the user to enter the name of their insurance provider into an insurance company name field 1018, followed by the name of the user's employer or group in an employer or group name field 1020. The avatar may further prompt the user to input the primary policyholder's name in a subscriber name field 1022, provide the subscriber's date of birth in a subscriber date of birth field 1024, and enter the policy identifier in an ID #field 1026.

The backend system supporting the insurance information window 1016 can implement data validation services that check the format and structure of submitted data before it is committed to the patient intake database. For example, the system can verify that the date of birth field follows a predefined format, cross-reference the insurance company name against a database of recognized providers, or check the length and character structure of the policy number. In some implementations, backend services can also attempt to automatically match entered insurance details with existing patient records or insurance provider APIs, reducing the need for manual verification. The avatar 1004 can respond dynamically based on validation results—prompting the user to correct an error, providing clarifying instructions, or moving the workflow forward once all fields are properly completed.

FIG. 10C illustrates an insurance card window 1028, which is configured to collect images of the user's physical insurance card. This functionality enables the system to supplement text-based insurance data with visual documentation, which can be used for verification, claims processing, or recordkeeping. The insurance card window 1028 includes a first element 1030 that, if selected, causes the application to access the device's camera, thereby allowing the user to capture an image of the front side of the insurance card. A second element 1032 is similarly configured for capturing an image of the back side of the card. The avatar 1004 can continue to assist in this stage by explaining how to properly align the card within the camera frame, confirming if the captured image is clear and legible, and prompting the user to retake the photo if necessary.

On the backend, captured images can undergo several processing and storage operations. The application can automatically compress and format the images before uploading them to a secure cloud storage service. Metadata, such as timestamps, device identifiers, and associations with the patient's record, can be generated and stored alongside the images. In some implementations, the backend may perform OCR on the uploaded images to extract text information, such as policy numbers or provider names, and cross-check it against the data previously entered in the insurance information window 1016. These backend processes allow the system to ensure the accuracy and completeness of insurance information while reducing the need for manual review by administrative staff.

Taken together, FIGS. 10A-10C illustrate an example of a continuous, avatar-guided digital intake workflow designed to collect patient information in a structured and user-friendly manner. The process begins with the personal information window 1002 (FIG. 10A), where the user is guided by the AI-assisted avatar 1004 to provide basic identifying details such as name, contact information, and medical record number. The workflow then transitions to the insurance information window 1016 (FIG. 10B), where the avatar continues to assist by prompting for key insurance data, validating entered information, and ensuring data completeness. Following that, the user is directed to the insurance card window 1028 (FIG. 10C), where the application collects photographic documentation of the insurance card to support verification and recordkeeping. Throughout each stage, the avatar 1004 can interpret natural language commands, respond dynamically to user input, and adapt its behavior based on session state and validation results. On the backend, data collected from each window is validated, normalized, and securely stored, with optional services such as OCR processing and database cross-referencing enhancing the reliability and utility of the submitted information. Together, these interfaces form a cohesive, AI-driven intake system that improves data accuracy, streamlines patient onboarding, and reduces administrative workload compared to conventional manual or form-based approaches.

Embodiments, such as those discussed herein, may be directed at least in part to increasing efficiency and/or profitability of oral surgery practices, to name one example practice-type. In one or more embodiments, efforts may be aimed at elevating a platform's influence and/or to extend its market presence within an oral surgery sector, to again name but one example. In one or more embodiments, a bespoke CRM dashboard, tailored specifically for oral surgery practices, for example, may be developed. In one or more embodiments, such a dashboard may significantly improve patient engagement at (e.g., two, etc.) junctures, for example: transitioning from initial inquiry to appointment booking, and/or from consultation to the commencement of treatment, for example.

In one or more embodiments, an AI Avatar, referred to herein as “Aria,” may interact (e.g., seamlessly, etc.) with patients, simulating efficiency of in-person consultation during a virtual intake process, for example. Also, in one or more embodiments, functionalities such as, for example, text-to-speech, speech-to-text, and/or dynamic human avatars may be implemented, for example.

In one or more embodiments, a need has been observed for development of a CRM dashboard designed specifically for oral surgery practices. Of course, as mentioned, oral surgery practice is merely one example practice-type, and subject matter is not limited in scope in this respect. In one or more embodiments, VCI may facilitate identification and/or engagement of patients navigating through a referral pipeline, for example. In one or more embodiments, an objective may be to equip oral surgery practices with a streamlined mechanism to actively engage with referred patients, thereby reducing incidence of patient attrition at particular stages of a treatment process, for example. Such drop-off points may include, for example, Inquiry-to-Consult and/or Consult-to-Surgery. Such stages have been identified as pivotal phases where patient engagement may significantly impact continuity of care and/or likelihood of proceeding to surgery, for example.

In one or more embodiments, an objective of integrating CRM capabilities into a virtual consultation platform, such as VCI, for example, may be to enhance the bottom line of oral surgery practices by providing a streamlined solution to track, identify, and/or engage with patients at risk of dropping out during a referral process, for example. In one or more embodiments, such a strategic enhancement may be designed to reduce (e.g., minimize, etc.) patient disengagement and/or ensure continuous engagement throughout a patient's journey to surgery, for example.

In one or more embodiments, example core features may include, for example:
Inquiry-to-Consult: One or more embodiments may include an interactive pie/bar chart CRM that groups patients by workflow stages and/or allows a practice's administrator to send a blast notification reminder or to engage each patient individually, for example.
Consult-to-Surgery: One or more embodiments may include an interactive pie/bar chart CRM that groups patients into categories, such as, for example, financial, logistical, insurance and/or a custom category, for example. In one or more embodiments, this may enable a practice to engage each patient individually by category or send a blast email to all patients under each category, for example.
Daily Snapshot Report: In one or more embodiments, a daily email may be sent to a practice with a picture of charts (e.g., all charts, etc.) and their respective statistics to remind the practice of the number of referred patients that are going through their referral-to-surgery pipeline, for example.

### Patient View (Blast Messages)

In one or more embodiments, VCI may display email and/or text icons in the upper right corner (maybe three dots to put preset) of a dashboard, for example. In one or more embodiments, clicking on email icon may initiate an email notification process, for example. In one or more embodiments, clicking on the text icon may initiate the text message notification process, for example.

In one or more embodiments, VCI may provide a preset global message available for quick sending for each stage of a workflow, for example. In one or more embodiments, a preset message may be displayed in a pop-up message format if either notification method is selected, for example. In one or more embodiments, if a user selects either email or text notification, a pop-up window may appear displaying the preset messages, for example. In one or more embodiments, preset message may be customizable by an admin before sending it, for example. In one or more embodiments, if a user clicks on the preset button, they may be directed to a screen similar to a Message Manager, where preset messages may be displayed with their respective label, for example. In one or more embodiments, users may update messages by swiping left, delete messages by swiping right, and/or add new messages by clicking on the plus icon located in the lower right corner, for example. In one or more embodiments, if a preset message is selected, a user may click on a button labeled “Send” to dispatch the message, for example.

### Patient View (Individual Messages)

In one or more embodiments, a dashboard may display a list of patients, for example. In one or more embodiments, each patient entry may display the following example details: name and/or email. In one or more embodiments, a user may search for a specific patient through name, email and/or MR #, for example.

In one or more embodiments, if a user swipes left on a patient it may have an icon to send a message to the patient, for example. In one or more embodiments, this action may guide the user to a chat and at the upper right corner there may be options (e.g., three options, etc.), an icon for the text messages, another icon for the email and/or one where all the preset messages for that stage of the workflow, for example.

In one or more embodiments, if a user selects a preset message, they may have the option to choose whether the notification is sent via text message or email to the patient and then send it, for example.

### Status Update

In one or more embodiments, once a patient is on a final stage of a workflow (e.g., virtual intake process, etc.) and/or a note is submitted from a provider, a status of the patient on a database may change to “Completed,” for example. In one or more embodiments, this way the patient may no longer be a part of this dashboard, for example.

### Consult to Surgery

In one or more embodiments, VCI may display a pie chart representing a distribution of patients across different workflow stages, for example. In one or more embodiments, a pie chart may categorize patients into the following example types of issues: Financial (e.g., if patient has issues with payment, etc.), Logistical (e.g., if patient has issues going to a practice, etc.), Insurance (e.g., if patient has issues with insurance, etc.) and/or Custom (e.g., if patient has another type of issue, etc.), for example. In one or more embodiments, each piece of the pie chart may be segmented based at least in part on different percentages of active patients in each current issue, for example.

In one or more embodiments, VCI may update a pie chart in real-time as patients complete or change each issue, for example. In one or more embodiments, content (e.g., data, etc.) for a pie chart may be sourced from a patient management system database, for example.

In one or more embodiments, fields may be added to a “Users” database, for example. For example, a field named “currentIssue” may be added to designate a specific issue for each patient, and another field named “currentIssueTimestamp” may be added to record date and/or time if the patient does not have an issue or has another issue, for example.

In one or more embodiments, each piece of a pie chart may have a total number of patients that are on a specific issue, for example. In one or more embodiments, each tile may show a number of patients in that issue at a left side of the tile, for example. In one or more embodiments, below the chart, VCI may display tiles corresponding to each issue, for example. In one or more embodiments, users may be able to click on each tile to access to a patient list in that specific issue, for example.

### Patient View (Blast Messages)

In one or more embodiments, VCI may display email and/or text icons in an upper right corner (maybe three dots to put preset) of a dashboard, for example. In one or more embodiments, clicking on an email icon may initiate an email notification process, for example. In one or more embodiments, clicking on a text icon may initiate a text message notification process, for example.

In one or more embodiments, VCI may provide a preset global message available for quick sending for each issue, for example. In one or more embodiments, a preset message may be displayed in a pop-up message format if either notification method is selected, for example. In one or more embodiments, if a user selects either email or text notification, a pop-up window may appear displaying the preset messages, for example.

In one or more embodiments, a preset message may be customizable by an admin before sending it, for example. In one or more embodiments, if a user clicks on a preset button, they may be directed to a screen similar to a Message Manager, where preset messages may be displayed with their respective label, for example. In one or more embodiments, users can update messages by swiping left, delete messages by swiping right, and/or may add new messages by clicking on the plus icon located in a lower right corner.

In one or more embodiments, if a preset message is selected, a user can click on a button labeled “Send” to dispatch the message, for example.

### Patient View (Individual Messages)

In one or more embodiments, a dashboard may display a list of patients, for example. In one or more embodiments, each patient entry may display the following example details: name and/or email. In one or more embodiments, a user may search for a specific patient through name, email and/or MR #, for example.

In one or more embodiments, if a user swipes left on a patient it may have an icon to send a message to the patient, for example. In one or more embodiments, this action may guide a user to a chat and at an upper right corner there may be (e.g., three, etc.) options, an icon for text messages, another icon for email and/or one for preset messages for that type of issue, for example.

In one or more embodiments, if a user selects a preset message, they may have an option to choose whether a notification is sent via text message or email to a patient and then send it, for example.

In the preceding description, various aspects of claimed subject matter have been described. For purposes of explanation, specifics, such as amounts, systems and/or configurations, as examples, were set forth. In other instances, well-known features were omitted and/or simplified so as not to obscure claimed subject matter. While certain features have been illustrated and/or described herein, many modifications, substitutions, changes and/or equivalents will now occur to those skilled in the art. It is, therefore, to be understood that the appended claims are intended to cover all modifications and/or changes as fall within claimed subject matter.

What is claimed is:

1. An apparatus, comprising:
a processor coupled to a memory of at least one computing device, the processor to:
generate, via a graphical user interface (GUI), one or more digital signals comprising a digital representation of an n-dimensional model;
electronically generate a prompt to initiate generation of an online content, wherein the online content is to be partitioned according to one or more predefined aspects;
electronically assign one or more values corresponding to the one or more predefined aspects based, at least in part, on the electronic prompt; and
store the one or more assigned values as one or more digital signals in a particular database for use in one or more subsequent computing processes via the digital representation of the n-dimensional model.

2. The apparatus of claim 1, wherein the electronic prompt comprises at least one of the following: an audio prompt; an on-screen prompt; a haptic prompt; or any combination thereof.

3. The apparatus of claim 1, wherein the digital representation of the n-dimensional model comprises at least one of the following: a digital representation of a one-dimensional model; a digital representation of a multi-dimensional model; or any combination thereof.

4. The apparatus of claim 1, wherein the digital representation of the n-dimensional model comprises an on-screen representation of an Artificial Intelligence (AI)-assisted avatar.

5. The apparatus of claim 1, wherein the processor further to:
determine whether a particular digital signature is available; and
generate, responsive to the determination that the signature is not available, a signature prompt to capture the particular digital signature, wherein the particular digital signature is to be captured for use with the one or more subsequent computing processes.

6. The apparatus of claim 1, wherein the one or more subsequent computing processes includes one or more processes to initiate a transmission of the one or more assigned values to generate the online content partitioned according to the one or more predefined aspects.

7. The apparatus of claim 6, wherein the online content is to be generated to:
access the one or more assigned values to facilitate a prepopulation of the online content for the one or more subsequent processes;
initiate a workflow associated with an electronic intake process; and
generate an entry via an appointment management system based, at least in part, on the one or more assigned values.

8. The apparatus of claim 1, wherein the online content partitioned according to one or more predefined aspects is associated with a particular user profile.

9. A method, comprising:
generating, via a graphical user interface (GUI), one or more digital signals comprising a digital representation of an n-dimensional model;
electronically generating a prompt for generating an online content, wherein the online content is being partitioned according to one or more predefined aspects;
electronically assigning one or more values corresponding to the one or more predefined aspects based, at least in part, on the electronic prompt; and
storing the one or more assigned values as one or more digital signals in a particular database for use in one or more subsequent computing processes via the digital representation of the n-dimensional model.

10. The method of claim 9, wherein the electronic prompt comprises at least one of the following: an audio prompt; an on-screen prompt; a haptic prompt; or any combination thereof.

11. The method of claim 9, wherein the digital representation of the n-dimensional model comprises at least one of the following: a digital representation of a one-dimensional model; a digital representation of a multi-dimensional model; or any combination thereof.

12. The method of claim 9, wherein the digital representation of the n-dimensional model comprises an on-screen representation of an Artificial Intelligence (AI)-assisted avatar.

13. The method of claim 9, further comprising
determining whether a particular digital signature is available; and
generating, in response to determining that the particular digital signature is not available, a signature prompt to capture the particular digital signature, wherein the particular digital signature is captured for use with the one or more subsequent computing processes.

14. The method of claim 9, wherein the one or more subsequent computing processes comprises transmitting the one or more assigned values for generating the online content being partitioned according to the one or more predefined aspects.

15. The method of claim 14, wherein the one or more subsequent computing processes comprising transmitting the one or more assigned values for generating the online content further comprises:
accessing the one or more assigned values to facilitate a prepopulation of the online content for the one or more subsequent processes;
initiating a workflow associated with an electronic intake process; and
generating an entry via an appointment management system based, at least in part, on the one or more assigned values.

16. The method of claim 9, wherein the online content partitioned according to one or more predefined aspects comprises a particular user profile.

17. The method of claim 9, and further comprising accessing the one or more assigned values to generate electronic documentation in compliance with one or more privacy-regulated processes.

18. The method of claim 9, wherein the electronic prompt is generated in connection with one or more cues to direct attention to one or more progressive steps for generating the online content.

19. An article, comprising:
a computer-readable medium having stored thereon instructions executable by a computing device to:
generate, via a graphical user interface (GUI), one or more digital signals comprising a digital representation of an n-dimensional model;
electronically generate a prompt to initiate generation of an online content, wherein the online content is to be partitioned according to one or more predefined aspects;
electronically assign one or more values corresponding to the one or more predefined aspects based, at least in part, on the electronic prompt; and
store the one or more assigned values as one or more digital signals in a particular database for use in one or more subsequent computing processes via the digital representation of the n-dimensional model.

20. The article of claim 19, wherein the prompt comprises at least one of the following: an audio prompt; an on-screen prompt; a haptic prompt; or any combination thereof.

21. The article of claim 19, wherein the digital representation of the n-dimensional model comprises at least one of the following: a one-dimensional representation of an artificial intelligence (AI)-assisted avatar; a multi-dimensional representation of the AI-assisted avatar; or any combination thereof.

22. The article of claim 21, wherein the online content is to be generated to:
access the one or more assigned values to facilitate a prepopulation of the online content for the one or more subsequent processes;
initiate a workflow associated with an electronic intake process; and
generate an entry via an appointment management system based, at least in part, on the one or more assigned values.
